(19) United States
(12) Patent Application Publication
(10) Pub. No.: US 2009/0233917 A1
(43) Pub. Date: Sep. 17, 2009

(54) Stroke recovery

(76) Inventor: Jack R. Barber, San Diego, CA (US)

Correspondence Address: ROPES & GRAY LLP, PATENT DOCKETING 39/361, 1211 AVENUE OF THE AMERICAS, NEW YORK, NY, 10036-8704, US

(21) Appl. No.: 12/381,033

(22) Filed: Mar. 6, 2009

Related U.S. Application Data
(63) Continuation-in-part of application No. PCT/US2007/024711, filed on Nov. 30, 2007.
(60) Provisional application No. 60/872,329, filed on Dec. 1, 2006.
(60) Provisional application No. 60/920,396, filed on Mar. 27, 2007.
(60) Provisional application No. 60/993,848, filed on Sep. 14, 2007.

(51) Int. Cl. A61K 31/5395; A61K 31/4545; A61K 31/15; A61P 9/00

(52) U.S. Cl. 514/229.2; 514/318; 514/640

(57) ABSTRACT

The present invention provides methods of treating stroke comprising administering an effective amount of one or more of certain hydroxylamine derivatives to a subject in need thereof. The invention also provides pharmaceutical compositions comprising a certain hydroxylamine derivative or a pharmaceutically acceptable salt thereof, optionally in combination with one or more additional therapeutic agents. In certain compositions, the additional therapeutic agent is a second hydroxylamine derivative or a pharmaceutically acceptable salt thereof.

This application is a continuation-in-part and claims the benefit of International Application No. PCT/US2007/024711, filed Nov. 30, 2007 which claims the benefit of U.S. Provisional Application Nos. 60/872,329, filed Dec. 1, 2006; 60/920,396, filed Mar. 27, 2007; and 60/993,848, filed Sep. 14, 2007, the specification of each of which is incorporated by reference herein in its entirety. International Application No. PCT/US2007/024711 was published under PCT Article 21(2) in English.

### BACKGROUND OF THE INVENTION

Stroke is a medical emergency that affects about 700,000 persons per year in the United States alone. Stroke is caused by the sudden loss of blood supply to a part of the brain often followed by reperfusion, either naturally or by medical intervention. Both the lack of oxygen due to the lost blood supply and the reoxygenation upon reperfusion can cause death and/or damage to brain tissues of the affected area, often resulting in permanent and temporary paralysis or weakness on one half of the body, trouble seeing or speaking, problems with thinking, awareness, attention, learning, judgment and memory, emotional problems, and not uncommonly, death.

Stroke is categorized into two major types: ischemic stroke, which makes up more than 85% of all stroke incidents, and hemorrhagic stroke, which makes up the remaining events. In ischemic stroke, a blood vessel supplying oxygen to a part of a brain gets blocked, either by a clot developing at the location of blockage in an artery (thrombotic stroke), by a clot or plaque traveling to the site of blockage in an artery and lodging itself there (embolic stroke), or by a blockage of a vein, which results in impaired drainage, preventing fresh, oxygen-rich blood to enter into the affected area (venous thrombosis). In hemorrhagic stroke, a blood vessel ruptures or bleeds, resulting in the fresh blood not reaching the areas ahead of the breakage. In addition, with hemorrhagic stroke, the blood damages the brain tissue that it comes into contact with, as well as raising the intracranial pressure, especially if the drainage is blocked. Preferred treatments for ischemic stroke and hemorrhagic stroke may differ, especially with regard to the use of antithrombotic agents, because while a patient suffering from an ischemic stroke event may benefit from dissolving and removing the blood clot blocking the proper flow of blood, such agents may cause further bleeding and damage in a patient suffering from a hemorrhagic stroke event.

Ischemic injury in the brain leads to an increase in heat shock protein (HSP) expression in the brain tissue. HSPs are molecular chaperones, which is a class of proteins that play an essential role in a variety of cellular processes, mainly through assisting proper protein folding. For example, molecular chaperones bind noncovalently to nascent proteins and partially folded intermediates, and guide them along correct protein folding pathways, thereby preventing their irreversible aggregation and misfolding. Molecular chaperones also unfold proteins for their translocation across intracellular membranes into organelles. In addition, molecular chaperones facilitate the degradation of misfolded proteins.

Expression of HSP increases when a cell is exposed to elevated temperatures or other cellular stresses. HSPs are also referred to as “stress proteins” and their upregulation is sometimes described more generally as part of the “stress response”. It has been shown that pretreatment of animals with sub-lethal ischemia induces a molecular chaperone hsp70 expression and protects the brain against more severe subsequent ischemic insult; see Simon et al., Neurosci. Lett., 163:135-137 (1993), and against the burst of oxygen radicals that are generated when the blood flow is restored by rapid reperfusion.

Therefore, increased chaperone expression is thought to be protective and beneficial against ischemic and reperfusion injury.

Currently, there are three types of interventions to counter stroke: prevention of stroke, therapy immediately after the event to minimize the damages, and post-stroke rehabilitation. The effectiveness of a therapy to minimize damages decreases precipitously within an hour after the ischemic event. Because the likelihood of an access to such treatment within this short time is small unless the victim of stroke is already under medical surveillance, existing therapies of this type are of limited use.

Therefore, there is an unmet need for new methods of treating stroke. In particular, it would be desirable to find new treatment methods that minimize damages after stroke has already occurred, and to enhance the functional recovery of damaged brain tissue even when the treatment is administered starting one or more hours after stroke.

### SUMMARY

Methods of treating stroke are provided, comprising administering an effective amount of one or more of certain hydroxylamine derivatives to a subject in need thereof, e.g., a subject that was diagnosed as having suffered a stroke, demonstrating symptoms or surrogate markers associated with stroke, and/or suspected of having suffered stroke. Certain of the hydroxylamine derivatives useful for practicing the present methods include, but are not necessarily limited to, those previously described in U.S. Pat. No. 5,147,879; U.S. Pat. No. 6,143,741; U.S. Pat. No. 6,653,326; U.S. Pat. No. 6,649,628; U.S. Pat. No. 6,384,029; U.S. Pat. No. 5,328,906; U.S. Pat. No. 5,296,606; U.S. Pat. No. 5,919,796; U.S. Pat. No. 6,002,002; U.S. Pat. No. 6,180,787; U.S. Pat. No. 6,384,029; and U.S. Pub. 2005/0043295, which are incorporated by reference herein.

Exemplary compounds useful for practicing the present methods include: N-[2-hydroxy-3-(1-piperidinyl) propoxy]-3 pyridine-carboximidoyl-chloride (bimoclomol),

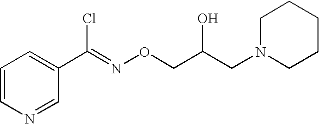

N-[2-hydroxy-3-(1-piperidinyl)propoxy]-pyridine-1-oxide-3-carboximidoyl chloride (arimoclomol),

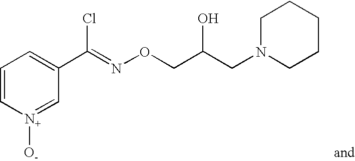

5,6-dihydro-5(1-piperidinyl)-methyl-3-(3-pyridyl)-4H-1,2,4-oxadiazine (iroxanadine).

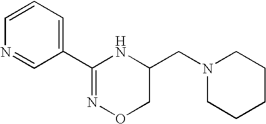

Another exemplary compound useful for practicing the present methods is N-[3-(1,1-dimethylethyl)amino] 2-hydroxypropoxy]-3-trifluoromethylbenzene-carboximidoyl chloride (Compound 2)

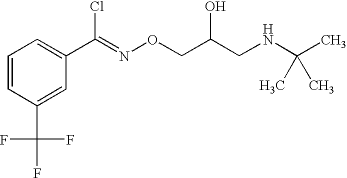

The formula for any of the above compound is intended to include all stereochemical forms of the compound, including geometric isomers (i.e., E, Z) and optical isomers (i.e., R, S). Single stereochemical isomers as well as enantiomeric and diastereomeric mixtures of the present compounds are within the scope of the present invention. Unless otherwise stated, formulae depicted herein are also meant to include compounds which differ only in the presence of one or more isotopically enriched atoms, and all salts of the foregoing.

Any of the above compounds may be used alone or in combination, and optionally in combination with one or more additional therapeutic agents for the treatment of a disease, disorder or condition in which molecular chaperones have been implicated. Preferred additional therapeutic agents are provided.

More generally, an embodiment of the present method may also be carried out using pharmaceutical compositions comprising a compound of Formula (I) or its tautomer compound of Formula (I):

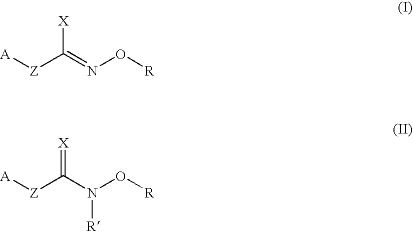

and pharmaceutically acceptable salts thereof, wherein, in each of compounds of Formulae (I) and (II):

A is an alkyl, substituted alkyl, aralkyl, aralkyl substituted in the aryl and/or in the alkyl moiety, aryl, substituted aryl, heteroaryl or substituted heteroaryl group;

Z is a covalent bond, oxygen or N(R³);

R³ is selected from the group consisting of hydrogen, an alkyl, substituted alkyl, aryl, substituted aryl, aralkyl, or aralkyl substituted in the aryl and/or in the alkyl moiety;

R is an alkyl or substituted alkyl,

X, in compound of Formula (I), is halogen or a substituted hydroxy or amino, monosubstituted amino or disubstituted amino group and, in compound of Formula (II), is oxygen, imino or substituted imino group;

R′ is hydrogen, an alkyl, substituted alkyl, aryl, substituted aryl, aralkyl, aralkyl having substituted aryl and/or alkyl moiety, acyl or substituted acyl group;

In one embodiment, the present methods comprise administering immediately after the stroke a hydroxylamine derivative to a subject that suffered from stroke. In another embodiment, the methods comprise administering the first dose of a hydroxylamine derivative at least 0.25, 0.5, 1, 2, 6, 24, 48, or 72 hours or more after the stroke.

In another embodiment, the present methods comprise administering one or more additional therapeutic agents in combination with one or more hydroxylamine derivatives. In a preferred embodiment, the method comprises administering the combination of arimoclomol and iroxanadine. In another embodiment, the additional therapeutic agent is a drug known to alleviate symptoms associated with stroke. In particular embodiments, the additional therapeutic agent is selected from anti-inflammatory agents, oxygen radical scavenger, anti-pyretic agents, anti-thrombosis agents (antiplatelet agents and anticoagulants), thrombolytics, and neuroprotective agents.

According to a preferred embodiment, the present pharmaceutical compositions are orally administered.

The present invention also provides pharmaceutical compositions comprising one or more hydroxylamine derivatives for the treatment of stroke.

### BRIEF DESCRIPTION OF THE FIGURES

FIG. 1 shows the functional recovery with the administration of hydroxylamine derivatives of rats with a permanent occlusion, shown as the improvement of forelimb placing test, as described herein as Example 1.

FIG. 2 shows the functional recovery with the administration of hydroxylamine derivatives of rats with a permanent occlusion, shown as the improvement of hindlimb placing test, as described in Example 1.

FIG. 3 shows the functional recovery with the administration of hydroxylamine derivatives of rats with a permanent occlusion, shown as the improvement of body swing test, as described in Example 1.

FIG. 4 shows the body weight of the experimental animals in the experiment described in Example 1.

FIG. 5 shows the infarct size over the course of treatment of the experiment as described in Example 1.

FIG. 6 shows the functional recovery with the administration of arimoclomol, a hydroxylamine derivative, to rats with a permanent occlusion, shown as an improvement in a forelimb placing test, as described in Example 4.

FIG. 7 shows the functional recovery with the administration of arimoclomol to rats with a permanent occlusion, shown as an improvement in a hindlimb placing test, as described in Example 4.

FIG. 8 shows the functional recovery with the administration of arimoclomol to rats with a permanent occlusion, shown as an improvement in a body swing test, as described in Example 4.

FIG. 9 shows the body weight of the experimental animals described in Example 4.

FIG. 10 shows a dose-response effect on the functional recovery with the administration of arimoclomol to rats with a permanent occlusion, shown as an improvement in a forelimb placing test, as described in Example 2.

FIG. 11 shows a dose-response effect on the functional recovery with the administration arimoclomol to rats with a permanent occlusion, shown as an improvement in a hindlimb placing test, as described in Example 2.

FIG. 12 shows a dose-response effect on the functional recovery with the administration of arimoclomol to rats with a permanent occlusion, shown as an improvement in a body swing test, as described in Example 2.

FIG. 13 shows the body weight of the experimental animals described in Example 2.

FIG. 14 shows the percent cell death after Oxygen/Glucose deprivation as describe in Example 10.

FIG. 15 shows the relative human and rat oral arimoclomol pharmacokinetic drug exposures.

FIG. 16 shows the cerebrospinal fluid (CSF) levels achieved at 3 hour and 6 hour time points after oral administration of increasing amounts of arimoclomol.

FIGS. 17a-b show functional recovery on administration of arimoclomol to post-embolic stroke rats as measured by improvement in the adhesive removal test, as described in Example 13.

FIGS. 18a-b show functional recovery on administration of arimoclomol to post-embolic stroke rats as measured by improvement in the foot-fault test, as described in Example 13.

FIGS. 19a-b show functional recovery on administration of arimoclomol to post-embolic stroke rats as measured by improvement in the mNSS test, as described in Example 13.

FIG. 20 shows the infarct volume for post-embolic stroke rats measured following treatment with arimoclomol for 28 days following embolic stroke, as described in Example 13.

### DETAILED DESCRIPTION OF THE INVENTION

### 1. Definitions

For convenience, certain terms employed in the specification, examples, and appended embodiments, are collected here. Unless defined otherwise, all technical and scientific terms used herein have the same meaning as commonly understood by one of ordinary skill in the art to which this invention belongs.

The articles “a” and “an” are used herein to refer to one or to more than one (i.e., to at least one) of the grammatical object of the article. By way of example, “an element” means one element or more than one element.

The term “including” is used herein to mean, and is used interchangeably with, the phrase “including but not limited to.”

The term “or” is used herein to mean, and is used interchangeably with, the term “and/or,” unless context clearly indicates otherwise.

The term “such as” is used herein to mean, and is used interchangeably, with the phrase “such as but not limited to.”

The terms “disorders” and “diseases” are used inclusively and refer to any deviation from the normal structure or function of any part, organ or system of the body (or any combination thereof). A specific disease is manifested by characteristic symptoms and signs, including biological, chemical and physical changes, and is often associated with a variety of other factors including, but not limited to, demographic, environmental, employment, genetic and medically historical factors. Certain characteristic signs, symptoms, and related factors can be quantitated through a variety of methods to yield important diagnostic information.

The term “prophylactic” or “therapeutic” treatment refers to administration to the subject of one or more of the subject compositions. If it is administered prior to clinical manifestation of the unwanted condition (e.g., disease or other unwanted state of the host animal) then the treatment is prophylactic, i.e., it contributes to protection of the host against developing the unwanted condition, whereas if administered after manifestation of the unwanted condition, the treatment is therapeutic (i.e., it is intended to diminish, ameliorate or prevent progression of the unwanted condition or side effects therefrom).

The term “therapeutic effect” refers to a local or systemic effect in animals, particularly mammals, and more particularly humans, caused by a pharmacologically active substance or substances. The term thus means any substance intended for use in the diagnosis, cure, mitigation, treatment or prevention of disease or in the enhancement of desirable physical or mental development and conditions in an animal or human. The phrase “therapeutically-effective amount” means that amount of such a substance that produces some desired local or systemic effect at a reasonable benefit/risk ratio applicable to any treatment. In certain embodiments, a therapeutically-effective amount of a compound will depend on its therapeutic index, solubility, and the like. For example, certain compounds useful in the practice of the present methods may be administered in a sufficient amount to produce a reasonable benefit/risk ratio applicable to such treatment.

The term “effective amount” refers to the amount of a therapeutic reagent that when administered to a subject by an appropriate dose and regimen produces the desired result.

A “subject” or “patient” to be treated by the present methods can mean either a human or non-human animal, preferably a mammal.

The term “subject in need of treatment for a disorder” is a subject diagnosed with that disorder, demonstrating symptoms or surrogate markers associated with the disorder, or is suspected of having that disorder.

Throughout this specification, the word “comprise” or variations such as “comprises” or “comprising” will be understood to imply the inclusion of a stated integer or groups of integers but not the exclusion of any other integer or group of integers.

The term “alkyl” refers to straight or branched, saturated aliphatic hydrocarbon containing 1 to 21 carbon atoms. “Short chain alkyl” refers to an alkyl group containing from 1 to 8 carbon atoms. Examples of short chain alkyl groups include, but are not limited to, methyl, ethyl, propyl, isopropyl, butyl, isobutyl, sec-butyl, pentyl, tert-pentyl, hexyl, heptyl, and octyl groups. Preferably, the short chain alkyl contains from 1 to 6 carbon atoms and is selected from the group consisting of methyl, ethyl, propyl, isopropyl, butyl, isobutyl, sec-butyl, pentyl, tert-pentyl, and hexyl-groups. “Long chain alkyl” refers to an alkyl group containing from 9 to 21 carbon atoms. Examples of long chain alkyl groups include, but are not limited to, nonyl, decyl, undecyl, dodecyl, tridecyl, tetradecyl, pentadecyl, hexadecyl, heptadecyl, octadecyl, nonadecyl, eicosyl and heneicosyl groups. Preferably the long chain alkyl contains from 9 to 17 carbon atoms and is selected from the group consisting of nonyl, decyl, undecyl, dodecyl, tridecyl, tetradecyl, pentadecyl, hexadecyl, and heptadecyl groups.

The term “cycloalkyl” refers to a monocyclic, non-aromatic, hydrocarbon ring system containing 3 to 8 carbon atoms. “Short cycloalkyl chain” refers to a cycloalkyl group containing from 3 to 8 carbon atoms. Examples include, but are not limited to, cyclopropyl, cyclobutyl, cyclopentyl, cyclohexyl, cycloheptyl and cyclooctyl groups. Preferably, the cycloalkyl group contains from 3 to 7 carbon atoms and is selected from the group consisting of cyclopropyl, cyclobutyl, cyclopentyl, cyclohexyl and cycloheptyl.

The term “aryl” refers to a mono- or polycyclic ring system which contains 6, 10, 12 or 14 carbons in which at least one ring of the ring system is aromatic. Examples of aryl ring systems include, but are not limited to, phenyl, naphthyl, pentalenyl, anthracenyl groups. Preferably, the aryl group is phenyl or naphthyl groups.

The term “aralkyl” refers to an alkyl group, wherein one or more hydrogen atoms of the alkyl group is replaced by one or more aryl radical. Examples of aralkyl groups include, but are not limited to, benzyl, benzhydryl, trityl, 1-phenyl-ethyl, 2-phenylethyl, 2-benzhydryl-ethyl, 3-phenylpropyl, 1-methyl-2-phenyl-ethyl, 1-phenylbutyl, 4-tritylbutyl, 1,1-dimethyl-2-phenylethyl, 4-phenylbutyl, 5-phenylpentyl, and 6-phenylhexyl-groups. Preferably, the aralkyl group is a lower alkyl group containing from 1 to 4 carbon atoms, substituted with a phenyl group. Preferred aralkyl groups include, but are not limited to, benzyl, 1-phenylethyl, 2-phenylethyl, and 1-methyl-2-phenylethyl groups.

The term “heterocyclic” refers to a mono ring system which contains 1 to 15 carbon atoms and 1 to 4 heteroatoms, in which the ring system may optionally contain unsaturated bonds but is not aromatic. Heteroatoms are independently sulfur, nitrogen, or oxygen. Examples include, but are not limited to, aziridinyl-, azetidinyl-, oxaziranyl-, pyrrolidinyl-, imidazolidinyl-, pyrazolidinyl-, perhydro-thiazolyl-, perhydro-isoxazolyl-, piperidinyl-, piperazinyl-, perhydro-pyrimidinyl-, perhydro-pyridazinyl-, morpholinyl-, perhydro-1H-azepinyl, oxazolyl, and isoxazolyl, oxadiazolyl (e.g. 1,2,4-oxadiazolyl- and others). Preferably, the heterocyclic ring is a 3-8 membered ring system. More preferably, the heterocyclic ring is a 5-8 membered ring system. More preferably, the heterocyclic ring is 5-6 membered ring, containing 1-2 oxygen atoms and 1-3 N-atoms.

The term “heteroaryl” refers to a mono- or polycyclic ring system which contains 1 to 15 carbon atoms and 1 to 4 heteroatoms, and in which at least one of the rings in the ring system is aromatic. Heteroatoms are sulfur, nitrogen or oxygen. Preferably, the heteroaryl group is an unsaturated, 3-8 membered ring. More preferably, the heteroaryl group is a 5-6 membered, 1-4 N-containing unsaturated hetero-monocyclic group. Examples include, but are not limited to, pyrrolyl, pyrrolinyl, imidazolyl, pyrazolyl, pyridyl group and its N-oxide, pirimidinyl, pyrazinyl, pyridazinyl, triazolyl, tetrazolyl, and dihydrotriazinyl. Preferably, the heteroaryl group is a polycyclic ring containing 1-5 N-atoms. Examples include, but are not limited to, indolyl, isoindolyl, indolizinyl, benzimidazolyl, quinolyl, isoquinolyl, indazolyl, benzotriazolyl, tetrazolopyridyl, tetrazolopyridazinyl, and dihydro-triazolopyridazinyl. Preferably, the heteroaryl group is a polycyclic ring containing an unsaturated ring, 1-2 oxygen atoms and 1-3 N-atoms. Examples include, but are not limited to, benzoxazolyl and benzoxadiazolyl. Preferably, the heteroaryl group is a monocyclic, 3-8 membered ring, more preferably 5-6 membered ring, containing 1-2 sulfur atoms and 1-3 N-atoms. Examples include, but are not limited to, thiazolyl, 1,2-thiazolyl, thiazolinyl, and thiadiazolyl. Preferably, the heteroaryl group is a monocyclic, 3-8 membered ring, more preferably 5-6 membered ring, containing one sulfur atom or one oxygen atom. Examples include, but are not limited to, thienyl and furanyl. Preferably, the heteroaryl is a bicyclic ring containing 1-2 sulfur atoms and 1-3 nitrogen atoms. Examples include, but are not limited to, benzothiazolyl and benzothiadiazolyl.

The term “acyl” group refers to an acyl group which might be a short chain alkanoyl (e.g., formyl, acetyl, propionyl, butyryl and the like), a short chain alkoxy-carbonyl (e.g., methoxy-carbonyl, ethoxy-carbonyl, propoxy-carbonyl, butoxy-carbonyl, tert-butoxy-carbonyl and the like), a short chain alkyl-sulphonyl (e.g., methyl-sulphonyl, ethyl-sulphonyl and the like), aryl-sulphonyl (e.g., phenyl-sulphonyl and the like), aroyl (e.g., benzoyl, naphthoyl and the like), aryl-(short chain alkanoyl) (e.g., phenyl-acetyl, phenyl-propionyl and the like), cyclo-(short chain alkyl)-(short chain alkanoyl) (e.g., cyclohexyl-acetyl and the like), aryl-(short chain alkoxy)-carbonyl (e.g., benzyloxy-carbonyl and the like), aryl-carbamoyl (e.g., phenyl-carbamoyl, naphthyl carbamoyl and the like), cycloalkyl-carbamoyl (e.g., cyclohexyl-carbamoyl and the like), hetero-monocyclic sulphonyl (e.g., thienyl-sulphonyl, furyl-sulphonyl and the like). Acyl group may be optionally substituted with 1-3 substituents as described above.

The term “ω-amino-alkyl” group refers to a short chain alkyl group containing a substituted N-atom in the ω-position of the alkyl chain and in which the alkyl chain is optionally substituted with one or more substituents, preferably with one or two halogen (e.g., chloro, bromo, fluoro, iodo), hydroxyl group or acylated hydroxyl group. Preferably, one or two short chain alkyl groups and the “alkyl” definition is the same as written above. The N-atom in the ω-position of the alkyl chain can be substituted with one or two short chain alkyl substituents, preferably methyl-, ethyl-, tert-butyl- and the like, with cycloalkyl carbamoyl- (e.g., cyclohexyl-carbamoyl- and the like). Preferably, the N-atom can be a part of a saturated heterocyclic group which contains 1-4 nitrogen atoms and is selected from the group consisting of aziridinyl, azetidinyl, oxaziranyl, pyrrolidinyl, imidazolidinyl, pyrazolidinyl, perhydro-thiazolyl, perhydro-isoxazolyl, piperidinyl, piperazinyl, perhydro-pyrimidinyl, perhydro-pyridazinyl, morpholinyl, and perhydro-1H-azepinyl. The N-atom in the ω-position can be substituted with an aryl group (e.g., phenyl and the like), and can be quaternarized by a short chain alkyl substituent or oxidized as well.

The term “halogen” refers to F, Cl, Br, or I.

The term “optionally substituted” aryl or alkyl refers to an aryl- or alkyl group having one or more substituents. Examples of substituents include, but are not limited to, cyano, hydroxyl, short chain alkyl (e.g., methyl, ethyl, propyl, isopropyl, butyl, isobutyl, sec-butyl, pentyl, tert-pentyl, hexyl, heptyl, octyl and the like), short chain alkoxy (e.g., methoxy, ethoxy, propoxy, isopropoxy, butoxy, isobutoxy, sec-butoxy, tert-butoxy, pentyloxy, tert-pentyloxy, hexyloxy and the like), aryl (e.g., phenyl, naphthyl, and the like), nitro, amino, mono-(short chain alkyl)-substituted amino (e.g., methyl, ethyl, propyl, isopropyl, tert-butyl)-amino and the like, di-(short chain alkyl)-substituted amino (e.g., dimethylamino, diethylamino, dipropylamino, diisopropylamino, dibutylamino, dipentylamino, dihexylamino and the like), monohalogen, dihalogen or trihalogen (short chain)-alkyl (e.g., chloromethyl, 2,2-dichloroethyl, trifluoromethyl and the like) or halogen atom (e.g. fluoro-, chloro-, bromo-, and iodine atom).

The term “bioavailable” means that at least some amount of a particular compound is present in the systemic circulation. Formal calculations of oral bioavailability are described in terms of an F value (“Fundamentals of Clinical Pharmacokinetics,” John G. Wegner, Drug Intelligence Publications; Hamilton, Ill. 1975). F values are derived from the ratio of the concentration of the parent drug in the systemic circulation (e.g., plasma) following intravenous administration to the concentration of the parent drug in the systemic circulation after administration by a non-intravenous route (e.g., oral). Therefore, oral bioavailability within the scope of the present invention contemplates the ratio or F value of the amount of parent drug detectable in the plasma after oral administration compared to intravenous administration.

The term “treating” or “treatment” is intended to mean mitigating or alleviating the symptoms a disease in a mammal, such as a human, or the improvement of an ascertainable measurement associated with a disease.

The term “patient” refers to an animal including a mammal (e.g., a human).

The term “pharmaceutically acceptable derivative” refers to any pharmaceutically acceptable salt, ester, or salt of such ester, of a compound of this invention or any other compound which, upon administration to a recipient, is capable of providing (directly or indirectly) a compound of this invention or a metabolite or residue thereof.

### 2. Embodiments

The instant invention relates to methods of treating stroke, such as embolic stroke or thrombotic stroke or both, comprising administering an effective amount of one or more of certain hydroxylamine derivatives to a subject in need thereof, i.e. a subject that was diagnosed as having suffered a stroke, demonstrating symptoms or surrogate markers associated with stroke, and/or is suspected of having suffered stroke, such as embolic stroke or thrombotic stroke or both. The hydroxylamine derivatives useful for practicing the present methods include those previously described in:

U.S. Pat. No. 5,147,879; U.S. Pat. No. 6,143,741; U.S. Pat. No. 6,653,326; U.S. Pat. No. 6,649,628; U.S. Pat. No. 6,384,029; U.S. Pat. No. 5,328,906; U.S. Pat. No. 5,296,606; U.S. Pat. No. 5,919,796; U.S. Pat. No. 6,002,002 U.S. Pat. No. 6,180,787; U.S. Pat. No. 6,384,029; and U.S. Pub. 2005/0043295, all of which are incorporated by reference herein.

In one embodiment, the present methods comprise the step of administering to a subject in need thereof N-[2-hydroxy-3-(1-piperidinyl) propoxy]-3 pyridine-carboximidoyl-chloride (bimoclomol):

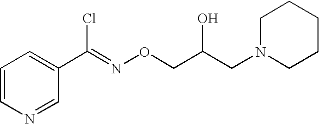

Bimoclomol was described in U.S. Pat. No. 5,147,879, which is incorporated herein by reference, and may be prepared by methods well known to those skilled in the art for analogous compounds. In particular, see U.S. Pat. No. 6,180,787, which is incorporated herein by reference.

In another embodiment, methods of the invention comprise the step of administering to a subject in need thereof N-[2-hydroxy-3-(1-piperidinyl)propoxy]-pyridine-1-oxide-3-carboximidoyl chloride (arimoclomol),

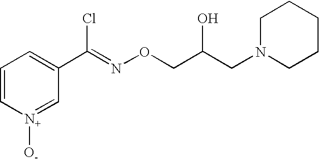

As described elsewhere, arimoclomol may be used to treat a patient having a disease, condition or disorder in which molecular chaperones have been implicated. Such diseases include, but are not limited to, neurodegenerative diseases. In some embodiments, the neurodegeneration is in the central nervous system (CNS). In some embodiments, the diseases are selected from the group consisting of stroke, Amyotrophic Lateral Sclerosis (ALS), Parkinson's Disease (PD), Alzheimer's Disease (AD), Huntington's Disease and cystic fibrosis.

Arimoclomol may be prepared by methods well known to those skilled in the art for analogous compounds. See, e.g., U.S. Pat. No. 6,649,628 and PCT Publication WO 01/79174, both of which are incorporated by reference herein.

Yet another embodiment of the invention comprises the step of administering to a subject in need thereof 5,6-dihydro-5(1-piperidinyl)-methyl-3-(3-pyridyl)-4H-1,2,4-oxadiazine (iroxanadine).

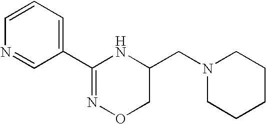

Iroxanadine and related compounds were previously described in PCT Publication WO 98/06400 and U.S. Pat. No. 6,384,029, which are incorporated by reference herein, and may be prepared by methods well known to those skilled in the art for analogous compounds, e.g., as described in these publications.

Another embodiment comprises the step of administering to a subject in need thereof N-[3-(1,1-dimethylethyl)amino-2-hydroxypropoxy]-3-trifluoromethylbenzene-carboximidoyl chloride (Compound 2)

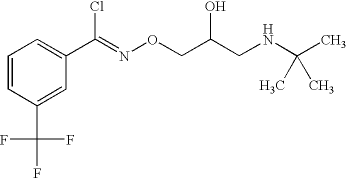

Compound 2 may be prepared by methods well known to those skilled in the art for analogous compounds. See, e.g., U.S. Pat. No. 6,649,628 and PCT Publication WO 01/79174, both of which are incorporated by reference. Compound 2 may be prepared, for example, using methods described for the preparation of arimoclomol in the above references, e.g., by starting with CF₃-cyanopyridine instead of CN-pyridine and substituting piperidine with tert-butylamine.

In particular embodiments, arimoclomol, iroxanadine, and Compound 2, alone or in combination with each other or with other therapeutic agents, are found to be effective in the treatment of stroke, such as embolic stroke or thrombotic stroke or both.

More generally, certain embodiments of the present invention may be carried out using pharmaceutical compositions comprising a compound of Formula (I) or its tautomer compound of Formula (II):

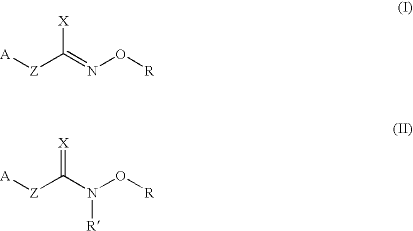

and pharmaceutically acceptable salts thereof, wherein, in each of compounds of Formulae (I) and (II):

A is an alkyl, substituted alkyl, aralkyl, aralkyl substituted in the aryl and/or in the alkyl moiety, aryl, substituted aryl, heteroaryl or substituted heteroaryl group;

Z is a covalent bond, oxygen or N(R³);

R³ is selected from the group consisting of hydrogen, an alkyl, substituted alkyl, aryl, substituted aryl, aralkyl, or aralkyl substituted in the aryl and/or in the alkyl moiety;

R is an alkyl or substituted alkyl,

X, in compound of Formula (I), is halogen or a substituted hydroxy or amino, monosubstituted amino or disubstituted amino group and, in compound of Formula (II), is oxygen, imino or substituted imino group; and

R′ is hydrogen, an alkyl, substituted alkyl, aryl, substituted aryl, aralkyl, aralkyl having substituted aryl and/or alkyl moiety, acyl or substituted acyl group.

The formula for any of the above compound is intended to include all stereochemical forms of the compound, including geometric isomers (i.e., E, Z) and optical isomers (i.e., R, S). Single stereochemical isomers as well as enantiomeric and diastereomeric mixtures of the present compounds are within the scope of the invention. Unless otherwise stated, formulae depicted herein are also meant to include compounds which differ only in the presence of one or more isotopically enriched atoms. For example, compounds having the present formulae except for the replacement of a hydrogen by a deuterium or tritium, or the replacement of a carbon by a ¹³C- or ¹⁴C-enriched carbon are within the scope of this invention.

In some embodiments, the compound of Formula (I) or (II) has the “R” configuration at the carbon containing the hydroxyl group. In some embodiments, the compound of Formula (I) or (II) has the “S” configuration at the carbon containing the hydroxyl group.

In some embodiments, the compound of Formula (I) or (II) has the “E” configuration across the carbon-nitrogen double bond. In some embodiments, the compound of Formula (I) or (II) has the “Z” configuration across the carbon-nitrogen double bond.

In one embodiment, in compounds of Formula (I), Z is a covalent bond and X is a halogen. In some aspects of this embodiment, X is chloro or bromo. In some aspects of this embodiment, A is selected from the group consisting of (i) aralkyl or aralkyl having substituted aryl moiety; (ii) aryl or substituted aryl; (iii) naphthyl; (iv) an N-containing heteroaryl group, including those which may be condensed with a benzene ring; (v) an S-containing heteroaryl group and (vi) an O-containing heteroaryl group. In some aspects of this embodiment, A is phenyl alkyl or phenyl alkyl having one or more substituents, preferably alkoxy. In other aspects of this embodiment, A is phenyl or substituted phenyl. In some aspects of this embodiment, A is substituted phenyl containing one or more substituents selected from the group consisting of alkyl, halo, haloalkyl, alkoxy and nitro. In some aspects of this embodiments, A is pyridyl. In further aspects of this embodiment, R is selected from the group consisting of (i) ω-amino-alkyl, (ii) ω-amino-alkyl having mono or disubstituted amino moiety; (iii) ω-amino alkyl having substituted alkyl moiety; (iv) ω-amino alkyl having mono or disubstituted amino moiety and also substituted alkyl moiety. In some aspects of this embodiment, when R is (iv), the alkyl group is substituted with a hydroxy or acyloxy group. In some aspects of this embodiment, the ω-amino-alkyl group is a 3-8 carbon atom alkyl moiety.

Compounds of Formula (I) in which Z is a covalent bond and X is a halogen are disclosed in U.S. Pat. Nos. 5,147,879, 5,328,906, and 5,296,606, all of which are incorporated by reference. These compounds can be prepared by procedures described in the cited U.S. patents, preferably by diazotization of the corresponding X═NH₂ derivatives in the presence of the appropriate hydrohalide. The starting compounds can be obtained by known procedures, e.g., those described in Hungarian Patent No. 177.578 (1976), namely by coupling an amidoxime of Formula (1) (R¹=R²=H):

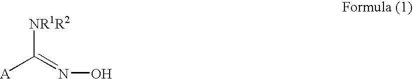

with e.g. a reactive derivative of Formula (2):

R-L Formula (2)

in the presence of a base, and can be diazotized usually without isolation or purification. The terminal groups A and R of the compounds can be further amidified or derivatized, as desired.

In another embodiment, in compounds of Formula (I), Z is covalent bond and X is a substituted hydroxy group O-Q, wherein Q is an unsubstituted or substituted alkyl or aralkyl group. In one aspect of this embodiment, Q is a straight or branched alkyl. In one aspect of this embodiment, A is aryl or heteroaryl; and R is selected from the group consisting of (i) ω-amino-alkyl, (ii) ω-amino-alkyl having mono or disubstituted amino moiety; (iii) ω-amino alkyl having substituted alkyl moiety; (iv) ω-amino alkyl having mono or disubstituted amino moiety and also substituted alkyl moiety. In some aspects of this embodiment, A is a N-containing heteroaromatic group. In some aspects of this embodiment, when R is (iv), the alkyl group is substituted with a hydroxy or acyloxy group. In some aspects of this embodiment, the ω-amino-alkyl group is a 3-8 carbon atom alkyl moiety.

In another embodiment, in the compound of Formula (I), Z is a covalent bond, X is O-Q, Z is a covalent bond, and R is a—CH₂—CH(OH)—R″. The compound is cyclized through the hydroxy group and is represented by Formula (I′):

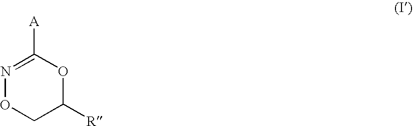

R″ is selected from the group consisting a straight or branched alkyl and a substituted straight or branched alkyl. In some aspects of this embodiment, R″ is ω-amino-alkyl which is optionally substituted on its amino group. In some aspects of this embodiment, R″ is ω-amino-alkyl which is substituted on its amino group with a C₁₋₅ straight or branched alkyl chain. In some aspects, R″ is ω-amino-alkyl mono- or disubstituted on the amino group, wherein the amino-substituents, independently from each other may be one or two straight or branched alkyl or cycloalkyl, or the two amino-substituents, together with the adjacent N-atom form a 3 to 7 heterocyclic ring. In some aspects, the ring is a 5 to 7-membered hetero ring, optionally containing an additional heteroatom. In some aspects, A is selected from the group consisting of phenyl, substituted phenyl, N-containing heteroaryl, substituted N-containing heteroaryl, S-containing heteroaryl, and substituted S-containing heteroaryl.

Compounds of Formula (I′) in which Z is a covalent bond and X is a O-Q are disclosed in Hungarian Patent Application No. 2385/1992, which is incorporated by reference. These compounds may be prepared from compounds of Formula (I) in which Z is covalent bond and X is halogen by procedures described in the Hung. Pat. Appln. No. 2385/1992 e.g., by reaction with alkoxides, or by alkaline ring closure for the cyclic compounds of Formula (I′).

In another embodiment, in the compounds of Formula (I), Z is a covalent bond and X is NR₁R₂, wherein R₁ and R₂ are independently selected from the group consisting of H, straight or branched alkyl, substituted straight or branched alkyl, cycloalkyl, or R₁ and R₂, together with the nitrogen atom to which they are bound, form a saturated ring containing 3 to 7 membered ring. In some aspects of this embodiment, R₁ and R₂ form a saturated 5-7 membered ring. In some aspects of this embodiment, R is selected from the group consisting of (i) ω-amino-alkyl, (ii) ω-amino-alkyl having mono or disubstituted amino moiety; (iii) ω-amino alkyl having substituted alkyl moiety; and (iv) ω-amino alkyl having mono or disubstituted amino moiety and also substituted alkyl moiety. In some aspects of this embodiment, when R is (iv), the alkyl group is substituted with a hydroxy or acyloxy group. In some aspects of this embodiment, the ω-amino-alkyl group is a 3-8 carbon atom alkyl moiety. In some aspects of this embodiment, A is selected from the group consisting of (i) aralkyl or aralkyl having substituted aryl moiety; (ii) aryl or substituted aryl; (iii) naphthyl; (iv) an N-containing heteroaryl group, including those which may be condensed with a benzene ring; (v) an S-containing heteroaryl group and (vi) an O-containing heteroaryl group. In some aspects of this embodiment, A is phenylalkyl or substituted phenylalkyl having one or more substituents. In some aspects of this embodiment, A is phenyl alkyl substituted by one or more alkoxy groups. In some aspects of this embodiment, A is phenyl or substituted phenyl. In some aspects of this embodiment, A is substituted phenyl containing one or more substituents selected from the group consisting of alkyl, halogen, haloalkyl, alkoxy, nitro, and acylamino group. In other aspects of this embodiment, A is pyridyl.

Compounds of Formula (I) in which Z is a covalent bond and X is NR¹R² are disclosed in Hungarian Patent No. 177578 (1976) and U.S. Pat. No. 6,653,326, both of which are incorporated by reference. These compounds may be synthesized by alkylation of unsubstituted amidoxime derivatives of compounds of Formula (I) (Formula (I) wherein R¹═R²═H) with a reactive derivative of compounds of Formula (II) in presence of a base.

In another embodiment, in the compound of Formula (I), Z is a covalent bond, X is NR₁R₂, and R is a—CH₂—CH(OH)—R″. The compound is cyclized through the NR₁R₂ group and is represented by Formula (I″):

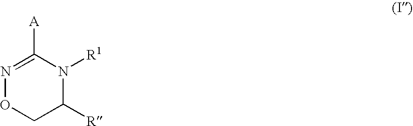

wherein R″ is selected from the group consisting of straight or branched alkyl or a substituted straight or branched alkyl. R¹ is selected from the group consisting of hydrogen, unsubstituted or substituted straight or branched alkyl, cycloalkyl, unsubstituted aralkyl and aralkyl substituted in the aryl- and alkyl moiety. In some aspects of this embodiment, A is selected from the group consisting of (i) aryl or substituted aryl; (ii) naphthyl; (iii) an N-containing heteroaryl group, including those which may be condensed with a benzene ring; (iv) S-containing heteroaryl group; and (v) O-containing heteroaryl group. In some aspects, A is phenyl or substituted phenyl. In some aspects, A is substituted phenyl containing one or more of alkyl, halogen, haloalkyl, alkoxy, amino or nitro group. In further aspects, R″ is selected from the group consisting of (i) ω-amino-alkyl having mono or disubstituted amino moiety, or (ii) ω-amino alkyl having mono or disubstituted amino moiety and also substituted alkyl moiety. In some aspects of this embodiment, the ω-amino-alkyl group is a 3-8 carbon atom alkyl moiety. In some aspects, the ω-amino-alkyl group has disubstituted amino moiety, wherein the substituents, together with the nitrogen atom attached thereto, form a saturated 3-7 membered heterocyclic ring. In some aspects, the ring is 5-7 membered and optionally contains an additional heteroatom. In some aspects, the ω-amino-alkyl groups the amino-substituent is a straight or branched alkyl group or cycloalkyl.

Compounds of Formula (I″) may be prepared by ring closure between atoms N(4)-C(5) using the open chain compound of Formula (I) in which Z is a covalent bond, X is ═NR¹R², wherein R¹ is as defined in connection with the compounds of the Formula (I″) above, R¹ is H, R is —CH₂—CHY⁵—R″, where Y⁵ is a leaving group, e.g., a halogen atom. Such derivatives may be obtained from the corresponding Y⁵═OH compounds with inorganic halogenating agents, e.g., thionyl chloride or phosphorus pentachloride. The halogenation may be carried out with or without an inert solvent e.g. benzene, chloroform, tetrahydrofurane etc., usually by boiling. After removing the excess of the reagent, e.g., by evaporation of the thionyl chloride, the crude halogen derivative may be cyclized—either after or with-out isolation or purification—by treatment with a strong base, e.g., potassium butoxide in t-butanol to give compound of Formula (I″), which is finally isolated and purified by standard procedures (extraction, recrystallization, etc.)

According to one embodiment, in the compound of Formula (I), Z is oxygen and X is O-Q, wherein Q is selected from the group consisting of alkyl, substituted alkyl, aralkyl, and substituted aralkyl having substituted aryl or substituted alkyl moiety. In some aspects of this embodiment, when A is alkyl or substituted alkyl, it contains 1-4 carbon atoms. In some aspects, A is selected from the group consisting of a C₁₋₄ alkyl or substituted alkyl, aralkyl and substituted aralkyl having substituted aryl or substituted alkyl moiety. In some aspects of this embodiment, R is selected from the group consisting of (i) ω-amino-alkyl, (ii) ω-amino-alkyl having mono or disubstituted amino moiety; (iii) ω-amino alkyl having substituted alkyl moiety; and (iv) ω-amino alkyl having mono or disubstituted amino moiety and also substituted alkyl moiety. In some aspects of this embodiment, when R is (iv), the alkyl group is substituted with a hydroxy or acyloxy group.

The compounds of Formula (I) in which Z is oxygen and X is O-Q may be prepared by the reaction of O-substituted hydroxylamines of Formula (6): (see e.g., Ger. Off. 2,651,083 (1976)) and orthoesters of Formula (7):

H₂N—O—R Formula (6)

C(OQ)₄. Formula (7)

The condensation may be carried out in the regent itself, as a solvent, preferably by boiling. After evaporation, the product may be isolated by crystallization, if there is an amine function in the side chain R, in the form of acid addition salt.

According to one embodiment, in the compound of Formula (I), Z is oxygen, X is NR¹R², and R¹ and R² are independently selected from the group consisting of H, a straight or branched alkyl, a substituted straight or branched alkyl, cycloalkyl, aryl, and substituted aryl, or R¹ and R², together with the nitrogen atom attached thereto, form a saturated ring containing 3 to 7 member saturated ring. In some aspects, R¹ and R² form a 5-7 membered saturated ring. In some aspects of this embodiment, R is selected from the group consisting of (i) ω-amino-alkyl, (ii) ω-amino-alkyl having mono or disubstituted amino moiety; (iii) ω-amino alkyl having substituted alkyl moiety; and (iv) ω-amino alkyl having mono or disubstituted amino moiety and also substituted alkyl moiety. In some aspects of this embodiment, when R is (iv), the alkyl group is substituted with a hydroxy or acyloxy group. In some aspects of this embodiment, the ω-amino-alkyl group is a 3-8 carbon atom alkyl moiety. In some aspects of this embodiment, A is selected from the group consisting of (i) alkyl or substituted alkyl; (iii) aralkyl or aralkyl having substituted aryl and/or substituted alkyl moiety; and (iv) aryl or substituted aryl. In some aspects of this embodiment, A is phenyl or substituted phenyl.

The compounds of Formula (I) may be prepared as described hereinbelow, wherein the methods depend on the nature of X, namely whether X is an unsubstituted amino (NH₂) or a substituted amino functionality.

Compounds of Formula (I) in which X is NH₂ may be prepared by the addition of hydroxylamine of Formula (6) to an organic cyanate of formula A-O—CN (see, e.g., Chem. Ber. 98, 144 (1965)). The reaction may carried out preferably in an inert organic solvent, usually at room temperature. The isolation often requires chromatographic purification.

Compounds of Formula (I) in which X is monosubstituted amino group (e.g., NHR¹) may be prepared from known haloformimidates of Formula (9):

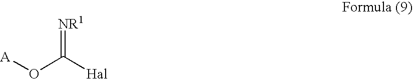

(see, e.g., Houben-Weil, “Methoden der Organischen Chemie,” Band E/4, p. 544 (1983) and a compound of Formula (6) in the presence of an organic base (e.g., triethylamine) or an inorganic base, such as sodium carbonate in an inert solvent, as benzene, tetrahydroturane, etc., followed by standard work-up and purification procedures.

Compounds of Formula (I) in which X is a disubstituted amino group may be prepared by the reaction of a secondary amine of Formula 5 with a compound of Formula (I), where Z is oxygen and X is O-Q (which may be prepared by the method described above):

HNR¹R² Formula (5)

These amination reactions are performed in polar organic solvents, e.g., ethanol, by refluxing, if necessary.

According to another embodiment, in the compound of Formula (I), Z is N(R³), wherein R³ is selected from the group consisting of hydrogen, an alkyl, substituted alkyl, aryl, substituted aryl, aralkyl, and aralkyl having substituted aryl or substituted alkyl moiety; and X is NR¹R², wherein R¹ and R² independently selected from the group consisting of H, a straight or branched alkyl, a substituted straight of branched alkyl, aryl or substituted aryl, cycloalkyl, and R¹ and R², together with the nitrogen atom attached thereto, form a 3 to 7 membered saturated ring.

In some aspects of this embodiment, A is selected from the group consisting of alkyl, substituted alkyl, aralkyl, aralkyl having substituted aryl or substituted alkyl moiety, aryl, and substituted aryl group. In some aspects, R¹ and R² form a 5-7 membered saturated ring. In further aspects of this embodiment, R is selected from the group consisting of (i) ω-amino-alkyl, (ii) ω-amino-alkyl having mono or disubstituted amino moiety; (iii) ω-amino alkyl having substituted alkyl moiety; and (iv) ω-amino alkyl having mono or disubstituted amino moiety and also substituted alkyl moiety. In some aspects of this embodiment, when R is (iv), the alkyl group is substituted with a hydroxy or acyloxy group. In some aspects of this embodiment, the ω-amino-alkyl group is a 3-8 carbon atom alkyl moiety.

Compounds of Formula (I) in which Z is NR³ and X is NR¹R², may be prepared by aminolysis of the corresponding isourea derivatives belonging to a group of compounds described above (i.e., compounds of Formula (I) in which Z is oxygen and X is NR¹R²) with ammonia or a primary or secondary amine. The reaction may be carried out preferably in a polar solvent, e.g., water or ethanol, using excess of the amine. Alternatively, haloformamides of Formula (10) (Houben-Weil “Methoden der Organischen Chemie,” Band E/4, page 553 (1983)) may be reacted with a compound having Formula (6) in the presence of an organic or inorganic base to give compounds of this group as well:

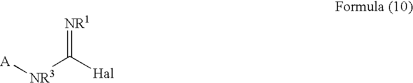

The reaction may be carried out in inert organic solvent, usually at ambient temperature.

Compounds of Formula (I) in which R is a group of the Formula (b):

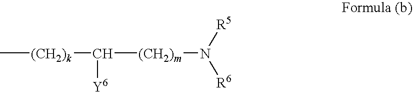

wherein R is acyl, may be prepared by esterifying the corresponding compounds containing hydrogen as R⁷. The alkyl or aryl esters may be obtained by using an acid chloride or anhydride in the presence of a tertiary amine or an inorganic base, preferably in an inert solvent.

It should be understood, however, that the group of compounds described above excludes hydroxylamine derivatives of the following structure:

R³-A-C(NH₂)═N—O—CH₂—CH(OH)—CH₂—N(R¹)(R²)

wherein

R¹ is H or C₁₋₅ alkyl,

R² is H, C₁₋₅ alkyl, C₃₋₈ cycloalkyl or phenyl which may be substituted with OH or phenyl, R¹ and R², when taken together with the adjacent nitrogen atom, form a 5-8 membered saturated or unsaturated ring, which optionally contains one or more additional N, O or S atom(s) and may be condensed with a benzene ring,

R³ is H or phenyl, naphthyl, or pyridyl optionally substituted with one or more halo or C₁₋₄ alkoxy,

A is a group of the formula (a),

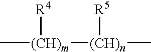

wherein

R⁴ is H or phenyl,

R⁵ is H or phenyl,

m is 0, 1 or 2, and

n is 0, 1 or 2.

According to another embodiment, the present invention provides compounds of Formula (II), which represents the tautomeric form of the compounds of Formula (I). In one aspect of this embodiment, in the compound of Formula (II), Z is covalent bond and X is oxygen. In further aspects of this embodiment, A is selected from the group consisting of (i) alkyl, aralkyl or aralkyl having substituted aryl or alkyl moiety; (ii) aryl or substituted aryl; (iii) an N-containing heteroaryl group; and (iv) S-containing heteroaryl group. In some aspects of this embodiment, A is phenyl or substituted phenyl having one or more substitutents. In some aspects of this embodiment, A is substituted phenyl containing one or more substituents selected from the group consisting of alkyl, haloalkyl and alkoxy. In other aspects of this embodiment, A is pyridyl.

In further aspects, R is selected from the group consisting of (i) ω-amino-alkyl, (ii) ω-amino-alkyl having mono or disubstituted amino moiety; (iii) ω-amino alkyl having substituted alkyl moiety; and (iv) ω-amino alkyl having mono or disubstituted amino moiety and also substituted alkyl moiety. In some aspects of this embodiment, when R is (iv), the alkyl group is substituted with a hydroxy or acyloxy group. In some aspects of this embodiment, the ω-amino-alkyl group is a 3-8 carbon atom alkyl moiety.

In further aspects, R′ is selected from the group consisting of hydrogen, an alkyl, substituted alkyl, aryl, substituted aryl, aralkyl, and aralkyl having substituted aryl or alkyl moiety.

Compounds belonging to this group are disclosed in the Hungarian Patent Application No. 2385/1992, which is incorporated by reference. These compounds may be prepared according to the methods described therein, most preferably, they can be obtained by acylation of O-substituted hydroxylamine derivatives having Formula (6) (see also, e.g., Ger. Off. 2,651,083 (1976)) with an acid chloride having Formula (11):

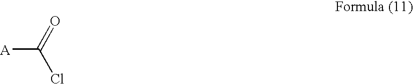

This route may also be employed for the preparation compounds in which R′ is other than hydrogen, using compound of Formula (12)—instead of Formula (6)—as starting material:

R¹HN—O—R Formula (12)

According to another embodiment, in compounds of Formula (II), Z is a chemical bond; X is ═NR⁴, wherein R⁴ is selected from the group consisting of H, an alkyl, substituted alkyl, aryl, substituted aryl, aralkyl, aralkyl having substituted aryl or substituted alkyl group, cycloalkyl; and R⁴ is selected from the group consisting of alkyl, substituted alkyl, aryl, substituted aryl, aralkyl, and aralkyl having substituted aryl or substituted alkyl moiety. In some aspects of this embodiment, A is (i) aralkyl or aralkyl having substituted aryl moiety; (ii) aryl or substituted aryl; (iii) naphthyl; (iv) an N-containing heteroaryl group; and (v) S-containing heteroaryl group. In some aspects of this embodiment, A is phenyl alkyl or phenyl alkyl having one or more substituents. In some aspects of this embodiment, A is phenyl alkyl substituted by one or more alkoxy groups. In some aspects of this embodiment, A is phenyl or substituted phenyl. In some aspects of this embodiment, A is substituted phenyl containing one or more substituents selected from the group consisting of alkyl, haloalkyl and nitro. In other aspects of this embodiment, A is pyridyl.

In some embodiments, R is selected from the group consisting of (i) ω-amino-alkyl, (ii) ω-amino-alkyl having mono or disubstituted amino moiety; (iii) ω-amino alkyl having substituted alkyl moiety; and (iv) ω-amino alkyl having mono or disubstituted amino moiety and also substituted alkyl moiety. In some aspects of this embodiment, when R is (iv), the alkyl group is substituted with a hydroxy or acyloxy group. In some aspects of this embodiment, the ω-amino-alkyl group is a 3-8 carbon atom alkyl moiety.

These compounds may be prepared either by O-alkylation of a N,N′-disubstituted amidoxime of Formula (13):

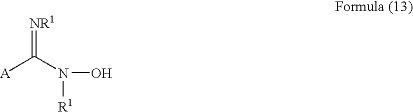

with a chemical compound having Formula (2) (for the reaction conditions, see preparation of compounds of Formula (I), wherein Z is covalent bond and X is NR¹R²), or by O-acylating an N,O-disubstituted hydroxylamine of the Formula (12) with an imidoyl halide of the Formula (16):

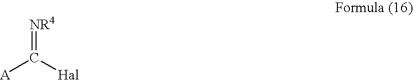

The reaction may be carried out in an inert solvent, preferably in the presence of an organic or inorganic acid scavenger.

The compounds wherein R is a group of the Formula (b) wherein R is acyl, may be prepared by esterifying the corresponding compounds containing hydrogen as R⁷. The alkyl or aryl esters may be obtained by using an acid chloride or anhydride in the presence of a tertiary amine or an inorganic base, preferably in an inert solvent.

According to one embodiment, in compounds of Formula (II), Z is oxygen and X is oxygen. In some aspects of this embodiment, A is selected from the group consisting of alkyl, substituted alkyl, aralkyl, and aralkyl with substituted aryl or alkyl moiety. In some aspects, R is selected from the group consisting of (i) ω-amino-alkyl, (ii) ω-amino-alkyl having mono or disubstituted amino moiety; (iii) ω-amino alkyl having substituted alkyl moiety; and (iv) ω-amino alkyl having mono or disubstituted amino moiety A) and also substituted alkyl moiety. In some aspects of this embodiment, when R is (iv), the alkyl group is substituted with a hydroxy or acyloxy group. In some aspects of this embodiment, the ω-amino-alkyl group is a 3-8 carbon atom alkyl moiety. In some aspects of this embodiment, R′ is selected from the group consisting of hydrogen, an alkyl, substituted alkyl, aryl, substituted aryl, aralkyl, and aralkyl with substituted aryl or alkyl moiety.

According to this embodiment, the compounds are disclosed in Hung. Patent Application No. 1756/95 (filed Jun. 15, 1995), which is incorporated by reference. These compounds may be prepared by acylation of a hydroxylamine having, Formula (6) or Formula (12) with a chloroformate having Formula (14), in a similar manner as with the simple acid chlorides, as described for the synthesis of compounds of Formula (II) wherein Z is covalent bond and X is oxygen. The reaction requires the presence of a base, inorganic or organic, and may be performed in an inert solvent, e.g., in chloroform. The side-product salt is removed, e.g., by extraction with water, and the product is isolated from the organic solution.

In yet another embodiment, in the compounds of Formula (II), Z is oxygen; X is ═NR⁴, wherein R⁴ is selected from the group consisting of alkyl, substituted alkyl, aralkyl, substituted aralkyl having substituted aryl or substituted alkyl group, aryl, substituted aryl, heteroaryl and substituted heteroaryl group. In some aspects of this embodiment, A is selected from the group consisting of alkyl, substituted alkyl, aryl, substituted aryl, aralkyl and aralkyl with substituted aryl or alkyl moiety. In some aspects of this embodiment, A is an unsubstituted or substituted phenyl.

In some aspects of this embodiment, R is ω-aminoalkyl, which suitably contains a hydroxy or acyloxy group in the alkyl chain, and is optionally substituted on the amine nitrogen, wherein the alkyl chain of the ω-aminoalkyl group preferably contains 3 to 8 carbon atoms. In some aspects of this embodiment, R′ is selected from the group consisting of alkyl, aryl or aralkyl which groups may be unsubstituted or substituted.

According to this embodiment, these compounds of Formula (I), wherein Z is oxygen and X is NR¹R² may be prepared, similarly from haloformimidates having Formula (9) and a chemical compound having Formula (12), in the presence of an organic base (e.g., triethylamine) or inorganic base, e.g sodium carbonate in an inert solvent, as benzene, tetrahydrofurane etc., followed by standard work-up and purification procedures.

In another embodiment, in the compounds of Formula (II), Z is N(R³), wherein R³ is selected from the group consisting of hydrogen, an alkyl, substituted alkyl, aryl, substituted aryl, aralkyl, and aralkyl having substituted aryl or substituted alkyl moiety; and X is oxygen. In some aspects of this embodiment, A is selected from the group consisting of (i) aralkyl or aralkyl having substituted alkyl or aryl moiety; (ii) aryl or substituted aryl, (iii) an N-containing heteroaryl group; (iv) an alkyl or substituted alkyl, straight or branched; and (v) a cycloalkyl group. In some aspects of this embodiment, A is phenyl alkyl or phenyl alkyl having one or more substituents. In some aspects of this embodiment, A is phenyl or substituted phenyl. In some aspects of this embodiment, A is substituted phenyl containing one or more substituents selected from the group consisting of alkyl, alkoxy, halogen, haloalkyl and nitro group. In other aspects of this embodiment, when a is (iv), the alkyl group contains 4 to 12 carbon atoms.

In some aspects of this embodiment, R is selected from the group consisting of (i) ω-amino-alkyl, (ii) ω-amino-alkyl having mono or disubstituted amino moiety; (iii) ω-amino alkyl having substituted alkyl moiety; and (iv) ω-amino alkyl having mono or disubstituted amino moiety and also substituted alkyl moiety. In some aspects of this embodiment, when R is (iv), the alkyl group is substituted with a hydroxy or acyloxy group. In some aspects of this embodiment, the ω-amino-alkyl group is a 3-8 carbon atom alkyl moiety. In some aspects, R¹ is selected from the group consisting of hydrogen, an alkyl, substituted alkyl, aralkyl, aralkyl having substituted aryl or alkyl moiety, aryl, substituted aryl, acyl and substituted acyl group.

According to this embodiment, these compounds are disclosed in a Hungarian Patent Application No. 1756/95, which is incorporated by reference, and may be prepared by the reaction of a hydroxylamine compound having Formula (6) or Formula (12) with an isocyanate having Formula (15):

A-N═C═O Formula (15)

in an inert solvent, usually by simple stirring of the mixture at room temperature for 2-24 hours. Finally, the products may be isolated, following evaporation of the solvent. In some aspects, the product may be isolated by crystallization.

In another embodiment, in the compounds of Formula (II), Z is N(R³), wherein R³ is selected from the group consisting of hydrogen, an alkyl, substituted alkyl, aryl, substituted aryl, aralkyl, and aralkyl having substituted aryl or substituted alkyl moiety; X is ═NR⁴, wherein R⁴ is selected from the group consisting of H, an alkyl, substituted alkyl, aryl, substituted aryl, aralkyl, aralkyl having substituted aryl or substituted alkyl group, and cycloalkyl; and R′ is selected from the group consisting of alkyl, substituted alkyl, aralkyl, substituted aralkyl having substituted aryl or substituted alkyl moiety, aryl and substituted aryl. In some aspects of this embodiment, R³ is selected from the group consisting of hydrogen, alkyl and substituted alkyl; R⁴ is hydrogen or an aryl group; and A is selected from the group consisting of alkyl, substituted alkyl, aryl and substituted aryl, or aralkyl, which may be substituted in the aryl and/or alkyl moiety. In further aspects, R is selected from the group consisting of (i) ω-amino-alkyl, (ii) ω-amino-alkyl having mono or disubstituted amino moiety; (iii) ω-amino alkyl having substituted alkyl moiety; and (iv) ω-amino alkyl having mono or disubstituted amino moiety and also substituted alkyl moiety. In some aspects of this embodiment, when R is (iv), the alkyl group is substituted with a hydroxy or acyloxy group. In some aspects of this embodiment, the ω-amino-alkyl group is a 3-8 carbon atom alkyl moiety.

According to this embodiment, the compounds may be prepared by aminolysis of the corresponding isourea derivatives (compounds of Formula (II), wherein Z is oxygen and X is NR⁴) with a primary or secondary amine or ammonia. The reaction may be carried out preferably in a polar solvent, e.g., water or ethanol, using an excess of the amine. Alternatively, the compounds may be prepared by reacting haloformamidines of Formula (10) with a compound of Formula (12) in the presence of an organic or inorganic base in inert solvents, usually at their boiling point.

According to one embodiment, the present invention provides compounds of Formula (I) in which X is halogen; Z is a chemical bond and A is a group of the Formula (a) wherein Y¹ is selected from the group consisting of halo, alkoxy, a nitro group and a haloalkyl group; and n is selected from the group consisting of 1, 2, and 3; or O-containing heteroaryl, S-containing heteroaryl, or N-containing heteroaryl group which may be condensed with a benzene ring; and R is a group having Formula (b), wherein R⁵ and R⁶, independently from each other, are selected from the group consisting of H, a straight or branched alkyl, and cycloalkyl, or R⁵ and R⁶, when taken together with the nitrogen atom attached thereto, form a 3 to 7; Y⁶ is —OR⁷ wherein R⁷ is H or an acyl group; k is 1, 2 or 3; and m is 1, 2, or 3, with the proviso, that when A is pyridyl or naphthyl, or a group of the Formula (a) wherein Y¹ is halo or alkoxy, then R⁷ is other than H. These compounds may optionally contain as A an N-containing heteroaromatic group with N-quaternary C₁₋₄ alkyl or the oxide of the said N-containing heteroaromatic group and/or an R wherein the ring formed by the terminal groups R⁶ and R⁷ is an N-quaternary or N-oxidized saturated heterocyclic ring.

In some aspects of this embodiment, X is chloro or bromo. In some aspects of this embodiment, Y¹ is haloalkyl containing 1-4 carbon atoms. In other aspects, Y¹ is selected from the group consisting of furyl, thienyl, pyridyl, quinolyl, and isoquinolyl. In some aspects of this embodiment, R⁵ and R⁶, independently from each other, is substituted straight or branched alkyl. In some aspects, R⁵ and R⁶ is C₁₋₄ alkyl. In other aspects, when R⁵ and R⁶ together with the nitrogen atom attached thereto form a 3 to 7, the resulting ring is a 5 to 7-membered saturated heterocyclic ring. In some aspects, R⁷ is selected from the group consisting of alkyl carbonyl, substituted alkyl carbonyl, aryl carbonyl or substituted aryl carbonyl, and aminoacyl or substituted aminoacyl.

In some aspects of this embodiment, A is a group of the Formula (a) wherein Y¹ is trifluoromethyl. In some aspects of this embodiment, X is halo, A is pyridyl, Z is a chemical bond, and R is the group of the Formula (b) wherein R⁵ and R⁶ independently from each other are selected from the group consisting of H, straight or branched alkyl, and cycloalkyl, or R⁵ and R⁶ together with the adjacent N atom form a 3 to 7-membered, Y⁶ is —OR⁷, wherein R⁷ is aminoacyl, k is 1, 2 or 3 and m is 1, 2 or 3. In some aspects, R⁵ and R⁶ independently from each other are C₁₋₄ alkyl or cycloalkyl. In other aspects, R⁵ and R⁶ together with the adjacent N atom form y 5 to 7-membered heterocyclic ring. According to each aspect of this embodiment, the compounds may be optically active.

According to this embodiment, these compounds may be prepared using procedures that are analogous to those described in U.S. Pat. Nos. 5,147,879: 5,398,906; and 5,996,606, all of which are incorporated by reference. For example, compounds in which both R⁵ and R⁶ are other than hydrogen, may be prepared by the diazotization of the corresponding NH₂ derivatives (i.e., the compound of Formula (I) in which Z is covalent bond and X is NH₂) in the presence of the appropriate hydrogen halide, similarly to the procedure described in U.S. Pat. Nos. 5,147,879; 5,328,906, and 5,296,606. The starting compounds may be obtained also by a known procedure, e.g., those described in Hungarian Patent No. 177578, which is incorporated by reference, namely by coupling an amidoxime having Formula (1), wherein R¹ and R² of Formula (1) is H, with e.g., a reactive derivative having Formula (2) in the presence of a base, and may be diazotized usually without isolation or purification.

Alternatively, for compounds in which R⁷ is H and m is 1, the compounds may be prepared by the reaction of an oxyrane of Formula (3) and amine of Formula (4). This procedure also may be used for the preparation of compound in which R⁵ is H.

Alternatively, for compounds in which R is represented by Formula (b) and R⁷ is an acyl group, the compounds may be prepared by the esterification of the corresponding compounds in which R⁷ is H. Alkyl or aryl esters may be obtained with an acid chloride or anhydride in the presence of a tertiary amine or an inorganic base, preferably in an inert solvent, or in certain cases by the Schotten-Bauman procedure using aqueous inorganic base in a two-phase system. For the preparation of the aminoacyl esters, carboxyl-activated N-protected amino acid derivatives (e.g., active esters) may be used as reagents in procedures basically known from the peptide chemistry. This coupling also requires the presence of a base (e.g., triethylamine). The isolation and purification of the products may be performed by using standard preparative techniques; the final preparation may often be in the form of a salt with appropriate inorganic or organic acids. Starting from chiral amino acids, the products may be frequently diastereomers, possessing the second chiral center in the R group. During the isolation, these diastereomers often may separate, and the product may be obtained in stereo-pure form.

In yet another embodiment of compounds of Formula (I), Z is a chemical bond, X is halo; A is a group of the Formula (c) and R is a group of the Formula (d):

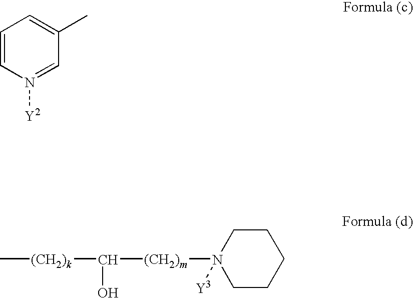

one or both of Y² and Y³ from which at least one must be present in the molecule, are oxygen, or an alkyl or substituted alkyl having 1-4 carbon atoms, k is 1, 2, or 3; and m is 1, 2, or 3. Y² and Y³ are attached by the dotted line. In some aspects of this embodiment, X is chloro or bromo. When the compound is a mono- or bivalent cation, the anion thereof is one or two halide ions. In some aspects of this embodiment, the anion is an iodide ion.

According to this embodiment, the compounds may be prepared by chemical modifications of the terminal pyridine and/or piperidine groups in their unsubstituted precurors, e.g., by N-oxidation or quaternization. In some aspects of this embodiment, the compounds may be prepared by oxidation with peracids in inert solvents. In further aspects of this embodiment, the peracid is a substituted perbenzoic acid. In further aspects of this embodiment, the inert solvent is chloroform or dichloromethane. If both oxidizable groups are present, mono- or dioxides may form depending on the quantity of the reagent used. At the end of the oxidation reaction, the excess reagent is decomposed and the product is isolated by evaporation. In other aspects of this embodiment, the compounds may prepared by quaternization. In some aspects of this embodiment, the compounds may be prepared by quarternization with alkyl halides. In some aspects of this embodiment, the alkyl halide is methyliodide. In further aspects of this embodiment, the compound may be prepared by refluxing the reagent in a suitable solvent. In some aspects, the solvent is acetone. In some aspects of this embodiment, the compound is insoluble in the medium, and may be isolated by simple filtration.

In yet another embodiment of compounds of Formula (I), Z is a chemical bond, A is selected from the group consisting of aralkyl, substituted aralkyl, phenyl, substituted phenyl having one or more substituents, a N-containing heteroaryl group, which may be condensed with benzene ring, and a sulfur containing heteroaromatic group; X is —NR¹R², wherein R¹ and R², independently from each other, are selected from the group consisting of H, a straight or branched alkyl, a substituted straight or branched alkyl, cycloalkyl and R¹ and R² taken together with the nitrogen atom attached thereto may form a 3 to 7; R is a group of the Formula (e)

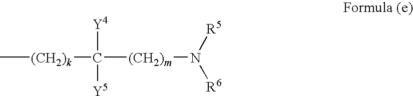

wherein R⁵ and R⁶, independently from each other, are selected from the group consisting of H, a straight or branched alkyl, or a substituted straight or branched alkyl, or cycloalkyl, or R⁵ and R⁶ taken together with the nitrogen atom attached thereto form a 3-7, which may contain additional hetero atoms and substituents; Y⁴ is selected from the group consisting of H, alkyl and substituted alkyl having 1-4 carbon atoms; Y⁵ is selected from the group consisting of H, alkyl and substituted alkyl; having 1-4 carbon atoms, or OR⁷, wherein R⁷ is H or an acyl; k is 1, 2, or 3; and m is 1, 2, or 3, with the proviso that when A is phenyl which is unsubstituted or substituted with halogen or alkoxy, or phenylalkyl substituted with alkoxy, or a pyridyl group, and R⁷ is H, then at least one of R¹ and R² is other than H, or when A is phenyl which is unsubstituted or substituted with halogen or alkoxy phenylalkyl substituted with alkoxy, or pyridyl, and R¹ and R² are each H, then R⁷ is other than H.

In some aspects of this embodiment, A is phenylalkyl or phenyl. In some aspects, when A is phenylalkyl, the phenyl may be substituted with one or more alkoxy groups. In some aspects, the alkoxy group has 1 to 4 carbon atoms. In other aspects, A is substituted phenyl having one or more substituents. In some aspects, the substituent groups are selected from the group consisting of an alkyl, preferably alkyl or haloalkyl having 1 to 4 carbon atom, halo, acylamino or nitro group. In other aspects, A is selected from the group consisting of pyrrolyl, pyridyl, isoquinolyl, quinolyl and thienyl. In some aspects, when A is a heteroaryl group, it may be substituted with one or more alkyl, preferably alkyl having 1 to 4 carbon atoms.

In some aspects of this embodiment, R¹ and R², independently from each other, are alkyl having 1 to 6 carbon atoms. In other aspects, when R¹ and R² are taken together with the nitrogen atom attached thereto form a ring, the ring is a 5-7 membered saturated hetero ring.

In some aspects of this embodiment, R⁵ and R⁶, independently from each other, are alkyl having 1 to 4 carbon atoms. In other aspects, when R⁵ and R⁶ are taken together with the nitrogen atom attached thereto to form a ring, the ring is a 5-7 membered saturated hetero ring, which may contain additional hetero atoms and substituents. In this aspect, the substituents may be alkyl having 1 to 4 carbon atoms.

According to this embodiment, compounds wherein X is NH₂ may be prepared, similarly to the above-mentioned procedure, by the reaction of the corresponding compound of Formula (1), wherein R¹ and R² of Formula (1) are H, with a compound of Formula 2. The alkylating agent of Formula 2 may contain hydroxyl and/or amino substituents. The reaction requires the presence of an inorganic or organic base, in a preferable manner alcoholic alcoholate solution is used as medium and base. The compounds may be isolated as a salt with a suitable organic or inorganic acid.

According to this embodiment, compounds wherein R¹ and R², one or both of them are other than H may be prepared by two methods. In the first method, an amidoxime of Formula (1), having the required substituents R¹ and/or R², may be reacted with a reactive compound of Formula (2), similarly to the procedure described in the previous paragraph. The substituted amidoximes of Formula (1), used as starting materials, are known from the literature. See, e.g., Chem. Rev. 62, 155-183 (1962), which is incorporated by reference.

In the second method, substitution of the halogen atoms in the compounds of Formula (I), wherein Z is covalent bond and X is halogen, by an amine of Formula (5) may result in similar compounds as well. In the case of derivatives bearing an OH substituent in the R group (Y⁴═OH), this hydroxyl group has to be protected before, and deprotected after the substitution reaction, otherwise formation of the cyclic derivatives of Formula (I′) is favored. For the protection, acetyl type protecting groups, e.g., tetrahydropyranyl group, have proven most satisfactory. The protection may be carried out by the reaction of the unprotected compound with dihydropyrane, followed by the halogen/amine displacement, which usually requires refluxing in a solvent, e.g., in alcohol. The deprotection of the product, finally, may be accomplished by acidic treatment, e.g., by boiling the ethanolic solution in the presence of e.g., p-toluenesulphonic acid.

According to another embodiment, compounds of Formula (I) include those wherein Y⁵ is an acyloxy group. They can be prepared by acylation of the corresponding compound in which Y⁵ is OH, which are either known from the literature (e.g, Hung. Patent No. 177578) or described in the present invention. The reactions may be accomplished identically to what is described for the analogous halo derivatives, wherein R⁷ is an acyl group.

According to another embodiment, compounds of Formula (I) also include those wherein Z is oxygen or an N(R³) group wherein R³ is an unsubstituted or substituted alkyl group; X is —NR¹R², wherein R¹ and R², independently from each other, are selected from the group consisting of hydrogen, unsubstituted or substituted straight or branched alkyl, unsubstituted or substituted aryl, and unsubstituted or substituted aralkyl group, or R¹ and R² are taken together with the nitrogen atom attached thereto to form a 3 to 7 membered saturated heterocyclic ring which optionally contains one or more hetero atoms. According to this embodiment, A is selected from the group consisting of an unsubstituted or substituted alkyl, an unsubstituted or substituted aryl, and unsubstituted or substituted aralkyl group. Further according to this embodiment, R is a group of the Formula (b) wherein R⁵ and R⁶, independently from each other are selected from the group consisting of H, straight or branched alkyl, and cycloalkyl, or R⁵ and R⁶ together with the N-atom attached thereto form a 3 to 7-membered saturated heterocyclic ring. According to this embodiment, Y⁶ is H or —OR⁷, wherein R⁷ is H or acyl, k is 1, 2 or 3 and m is 1, 2 or 3.

In one aspect of this embodiment, R¹ and R², independently from each other, are phenyl. In other aspects, when R¹ and R² are taken together with the nitrogen atom attached thereto to form a ring, the ring is a 5 to 7 membered saturated heterocyclic ring which optionally contains one or more heteroatoms. According to some aspects, A is phenyl or substituted phenyl group. According to some aspects, R⁵ and R⁶, independently from each other, are C₁₋₄ alkyl. Alternatively according to some aspects, R⁵ and R⁶ together with the N-atom attached thereto, form a 3 to 7-membered ring, the ring is a 5 to 7-membered saturated heterocyclic ring. According to some aspects, R⁷ is unsubstituted or substituted alkylcarbonyl or arylcarbonyl.

According to another embodiment, compounds of Formula (I) also include those wherein Z is oxygen and X is —OR^{X}, wherein Q is an unsubstituted or substituted alkyl or unsubstituted or substituted aralkyl group, A is an unsubstituted or substituted alkoxy group or an unsubstituted or substituted aralkyl group and R is a group of the Formula (b), wherein R⁵ and R⁶, independently from each other, are selected from the group consisting of H, straight or branched alkyl, and cycloalkyl, or R⁵ and R⁶, together with the N-atom attached thereto, form a 3 to 7-membered saturated heterocyclic ring, Y⁶ is H or —OR⁷, wherein R⁷ is H or acyl, k is 1, 2 or 3 and m is 1, 2 or 3.

In some aspects of this embodiment, R⁵ and R⁶, independently from each other, are C₁₋₄ alkyl. In other aspects, R⁵ and R⁶, when taken together with the N atom attached thereto form a 3 to 7-membered, the ring is a 5 to 7-membered heterocyclic ring. In some aspects, R⁷ is unsubstituted or substituted alkylcarbonyl or arylcarbonyl.

According to another embodiment, compounds of Formula (I) also include those wherein A is selected from the group consisting of unsubstituted or substituted aryl, N-containing heteroaromatic group and S-containing heteroaromatic group, Z is a chemical bond, X is —OQ wherein Q is C₁₋₄ alkyl and R is a group of the Formula (b), wherein R⁵ and R⁶, independently from each other are selected from the group consisting of H, straight or branched alkyl, and cycloalkyl, or R⁵ and R⁶, when taken together with the N atom attached thereto form a 3 to 7-membered heterocyclic ring, Y⁶ is H, k is 1, 2 or 3 and m is 1, 2 or 3.

In some aspects of this embodiment, A is phenyl. In other aspects, A is pyridyl. In some aspects of this embodiment, R⁵ and R⁶, independently from each other, are C₁₋₄ alkyl. In other aspects, R⁵ and R⁶ are taken together with the N atom attached thereto to form a 5 to 7-membered heterocyclic ring.

According to this embodiment, these compounds may prepared by the reaction of the corresponding compound of Formula (I) wherein X is halo and the corresponding alcoholates, preferably in an alcohol corresponding to the alcoholate, preferably by refluxing. The reaction mixture may be treated with methods known in the art and the product may be isolated by chromatography or salt-forming.

According to yet another embodiment, compounds of Formula (II) include those wherein X is oxygen, A is selected from the group consisting of C₁₋₂₀ straight or branched alkyl, unsubstituted or substituted aryl, unsubstituted or substituted aralkyl, naphthyl and N-containing heteroaromatic group, Z is a chemical bond, R′ is selected from the group consisting of H, C₁₋₄ alkyl and aralkyl, Z is a group of the Formula (b), wherein R⁵ and R⁶ independently from each other, are selected from the group consisting of H, straight or branched alkyl, and cycloalkyl, or R⁵ and R⁶ are taken together with the N atom attached thereto to form a 3 to 7-membered heterocyclic ring, Y⁶ is H or —OR⁷, R⁷ is H, k is 1, 2 or 3 and m is 1, 2 or 3, with the proviso, that when A is other than alkyl and R¹ is H, Y⁶ is H.

In some aspects of this embodiment, A is phenyl or halophenyl. In other aspects, A is pyridyl. In some aspects of this embodiment, R′ is phenylalkyl. In some aspects of this embodiment, R⁵ and R⁶ independently from each other, are C₁₋₄ alkyl. In other aspects, R⁵ and R⁶ are taken together with the N atom attached thereto to form a 5 to 7-membered heterocyclic ring.

According to yet another embodiment, compounds of Formula (II) also include those wherein Z is selected from the group consisting of a covalent bond, oxygen and an N(R³) group, wherein R³ is hydrogen or an unsubstituted or substituted alkyl group, X is ═NR⁴, wherein R⁴ is selected from the group consisting of hydrogen, an unsubstituted or substituted alkyl, an unsubstituted or substituted aryl, and a substituted or unsubstituted aralkyl. According to this embodiment, A is selected from the group consisting of an unsubstituted or substituted alkyl, an unsubstituted or substituted aryl, an unsubstituted or substituted aralkyl, and cycloalkyl, R′ is selected from the group consisting of an unsubstituted or substituted alkyl, an unsubstituted or substituted aryl, and an unsubstituted or substituted aralkyl, R is a group of the Formula (b), wherein R⁵ and R⁶, independently from each other, are selected from the group consisting of H, straight or branched alkyl, or R⁵ and R⁶ are taken together with the N atom attached thereto to form 3 to 7-membered heterocyclic ring, Y⁶ is H or —OR⁷, R⁷ is H or acyl, k is 1, 2 or 3 and m is 1, 2 or 3.

In some aspects of this embodiment, R⁴ is phenyl or phenylalkyl. In some aspects of this embodiment, A is selected from the group consisting of phenyl, substituted phenyl, and phenylalkyl. In some aspects of this embodiment, R′ is phenyl or phenylalkyl. In some aspects of this embodiment, R⁵ and R⁶, independently from each other, are C₁₋₄ alkyl. In other aspects, R⁵ and R⁶ are taken together with the N atom attached thereto to a form 5 to 7-membered heterocyclic ring. In some aspects of this embodiment, R⁷ is unsubstituted or substituted alkylcarbonyl or arylcarbonyl.

According to yet another embodiment, compounds of Formula (II) also include those wherein X is oxygen, A is unsubstituted or substituted alkyl, unsubstituted or substituted aralkyl, Z is oxygen, R′ is alkyl or aralkyl, preferably phenylalkyl, R is a group of the Formula (b), wherein R⁵ and R⁶, independently from each other, are selected from the group consisting of H, straight or branched alkyl, and cycloalkyl, or R⁵ and R⁶, when taken together with the N atom attached thereto form a 3 to 7-membered, Y⁶ is H or —OR⁷, R⁷ is H or acyl, k is 1, 2 or 3 and m is 1, 2 or 3. In some aspects, R⁵ and R⁶, independently from each other, are C₁₋₄ alkyl. In other aspects, R⁵ and R⁶ are taken together with the N atom attached thereto to form a 5 to 7-membered heterocyclic ring. In some aspects, R⁷ is unsubstituted or substituted alkylcarbonyl or arylcarbonyl.

In some aspects of this embodiment, A is phenylalkyl. In some aspects, R′ is phenylalkyl.

According to yet another embodiment, compounds of Formula (II) also include those wherein X is oxygen and Z is ═NH.

According to one embodiment, compounds of Formula (II) include those wherein A is selected from the group consisting of unsubstituted or substituted alkyl, cycloalkyl, and unsubstituted or substituted aralkyl, R is a group of the Formula (b), wherein R⁵ and R⁶, independently from each other, are selected from the group consisting of H, straight or branched alkyl, and cycloalkyl, or R⁵ and R⁶ are taken together with the N atom attached thereto to form a 3 to 7-membered heterocyclic ring, Y⁶ is H or —OH, k is 1, 2 or 3 and m is 1, 2 or 3.

In some aspects of this embodiment, A is phenylalkyl, unsubstituted phenyl or phenyl substituted with halo, alkyl, haloalkyl, alkoxy or nitro. In other aspects, R⁵ and R⁶, independently from each other, are C₁₋₄ alkyl. In other aspects, R⁵ and R⁶ are taken together with the N atom attached thereto to form a 5 to 7-membered heterocyclic ring.

According to one embodiment, compounds of Formula (II) include those wherein A is a group of the Formula (a):

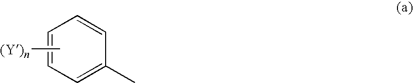

wherein Y¹ is haloalkyl, n is 1, 2 or 3, R′ is H and R is a group of the Formula (b), wherein R⁵ and R⁶, independently from each other, are selected from the group consisting of H, straight or branched alkyl, and cycloalkyl, or R⁵ and R⁶ are taken together with the N atom attached thereto to form a 3 to 7-membered heterocyclic ring, Y⁶ is H or —OH, k is 1, 2 or 3 and m is 1, 2 or 3.

In some aspects of this embodiment, Y¹ is trifluoromethyl. In other aspects, R⁵ and R⁶, independently from each other, are C₁₋₄ alkyl. In other aspects, R⁵ and R⁶ are taken together with the N atom attached thereto to form a 3 to 7-membered heterocyclic ring.

According to one embodiment, compounds of Formula (II) also include the cyclic compounds of the Formula (I″), wherein A is selected from the group consisting of unsubstituted phenyl, phenyl substituted with halo or nitro, and N-containing heteroaryl, R¹ is H and R″ is an ω-amino-alkyl group mono- or disubstituted on the amino group, the alkyl chain of which having 1 to 5 carbon atoms and the amino substituents, independently from each other, may be one or two straight or branched alkyl or cycloalkyl, or the two amino-substituents, together with the N atom adjacent thereto, form a 3 to 7-membered, preferably 5 to 7-membered saturated heterocyclic ring, or a C₁₋₄ alkyl N-quaternary derivative thereof, with the proviso, that when A is 3-pyridyl, R″ is different from 1-piperidinylmethyl.

Any of the above compounds may be used alone or in combination, optionally in combination with one or more additional therapeutic agents, for the treatment of a disease, disorder or condition in which molecular chaperones have been implicated. Such diseases include, but are not limited to, neurodegenerative diseases excluding diabetic peripheral neuropathies. In some embodiments, the neurodegeneration is in the central nervous system (CNS). In some embodiments, the diseases are selected from the group consisting of stroke, neurodegenerative diseases and cystic fibrosis. In some embodiments, the disease is stroke, such as embolic stroke or thrombotic stroke or both.

In certain embodiments, the present methods comprise administering immediately after the stroke a hydroxylamine derivative to a subject that has suffered from stroke, such as embolic stroke or thrombotic stroke or both. In other embodiments, the methods comprise administering the first dose of a hydroxylamine derivative at least 0.25, 0.5, 1, 2, 6, 10, 24, 48, or 72 hours or more after the stroke. In other embodiments, more than one hydroxylamine derivatives are ω-administered to a subject, either simultaneously or sequentially. In a preferred embodiment, the method comprises administering arimoclomol and iroxanadine.

In other embodiments, the present methods comprise administering one or more hydroxylamine derivatives to a subject that has suffered from stroke and one or more additional therapeutic agents. In another embodiment, the additional therapeutic agent is selected from anti-inflammatory agents, oxygen radical scavenger, anti-pyretic agents, anti-thrombosis agents (antiplatelet agents and anticoagulants), thrombolytics, neuroprotective agents, and EGFR inhibitors.

An anti-inflammatory and/or antipyretic agent may be: a non-steroidal anti-inflammatory (NSAIDs), such aminoarylcarboxylic acid derivatives such as enfenamic acid, etofenamate, flufenamic acid, isonixin, meclofenamic acid, mefanamic acid, niflumic acid, talniflumate, terofenamate and tolfenamic acid; arylacetic acid derivatives such as acemetacin, alclofenac, amfenac, bufexamac, cinmetacin, clopirac, diclofenac sodium, etodolac, felbinac, fenclofenac, fenclorac, fenclozic acid, fentiazac, glucametacin, ibufenac, indomethacin, isofezolac, isoxepac, lonazolac, metiazinic acid, oxametacine, proglumetacin, sulindac, tiaramide, tolmetin and zomepirac; arylbutyric acid derivatives such as bumadizon, butibufen, fenbufen and xenbucin; arylcarboxylic acids such as clidanac, ketorolac and tinoridine; arylpropionic acid derivatives such as alminoprofen, benoxaprofen, bucloxic acid; carprofen, fenoprofen, flunoxaprofen, flurbiprofen, ibuprofen, ibuproxam, indoprofen, ketoprofen, loxoprofen, miroprofen, naproxen, oxaprozin, piketoprofen, pirprofen, pranoprofen, protizinic acid, suprofen and tiaprofenic acid; pyrazoles such as difenamizole and epirizole; pyrazolones such as apazone, benzpiperylon, feprazone, mofebutazone, morazone, oxyphenbutazone, phenybutazone, pipebuzone, propyphenazone, ramifenazone, suxibuzone and thiazolinobutazone; salicylic acid derivatives such as acetaminosalol, aspirin, benorylate, bromosaligenin, calcium acetylsalicylate, diflunisal, etersalate, fendosal, gentisic acid, glycol salicylate, imidazole salicylate, lysine acetylsalicylate, mesalamine, morpholine salicylate, 1-naphthyl salicylate, olsalazine, parsalmide, phenyl acetylsalicylate, phenyl salicylate, salacetamide, salicylamine o-acetic acid, salicylsulfuric acid, salsalate and sulfasalazine; thiazinecarboxamides such as droxicam, isoxicam, piroxicam and tenoxicam; others such as ÿ-acetamidocaproic acid, s-adenosylmethionine, 3-amino-4-hydroxybutyric acid, amixetrine, bendazac, benzydamine, bucolome, difenpiramide, ditazol, emorfazone, guaiazulene, nabumetone, nimesulide, orgotein, oxaceprol, paranyline, perisoxal, pifoxime, proquazone, proxazole and tenidap; and pharmaceutically acceptable salts thereof; a steroidal antiinflammatory such as a glucocorticoid; and other analgesics, such as acetaminophen, and opiates.

Steroidal anti-inflammatory therapeutic agents (glucocorticoids) include, but are not limited to, 21-acetoxyprefnenolone, alclometasone, algestone, amicinonide, beclomethasone, betamethasone, budesonide, chloroprednisone, clobetasol, clobetasone, clocortolone, cloprednol, corticosterone, cortisone, cortivazol, deflazacort, desonide, desoximetasone, dexamethasone, diflorasone, diflucortolone, difluprednate, enoxolone, fluazacort, flucloronide, flumehtasone, flunisolide, fluocinolone acetonide, fluocinonide, fluocortin butyl, fluocortolone, fluorometholone, fluperolone acetate, fluprednidene acetate, fluprednisolone, flurandrenolide, fluticasone propionate, formocortal, halcinonide, halobetasol priopionate, halometasone, halopredone acetate, hydrocortamate, hydrocortisone, loteprednol etabonate, mazipredone, medrysone, meprednisone, methyolprednisolone, mometasone furoate, paramethasone, prednicarbate, prednisolone, prednisolone 25-diethylaminoacetate, prednisone sodium phosphate, prednisone, prednival, prednylidene, rimexolone, tixocortal, triamcinolone, triamcinolone acetonide, triamcinolone benetonide, triamcinolone hexacetonide, and pharmaceutically acceptable salts thereof.

An oxygen free radical scavenger may be: N-(2-mercaptopropionyl) glycine (MPG), edaravone, 2-(4-carboxyphenyl)-4,4,5,5-tetramethylimidazoline-1-oxyl-3-oxide (carboxy-PTIO), superoxide dismutase or its mimetic, or caffeic acid phenethyl ester (CAPE).

An antiplatelet agent may be: aspirin, clopidogrel or ticlopidine; it may be another combination drug such as Aggrenox (aspirin combined with extended-release dipyridamole);

An anticoagulant may be: warfarin, heparin, dalteparin (Fragmin), enoxaparin (Lovenox), or tinzaparin (Innohep).

An example of thrombolytic agents is tissue plasminogen activator (t-PA) or fondaparinux (Arixtra).

Other suitable agents to treat post stroke sequelae include glatiramer acetate, interferon β 1A, interferon β 1B, estradiol, progesterone, and mixtures thereof.

Suitable neuroprotectants include, but are not limited to donepezil, memanine, nimodipine, riluzole, rivastigmine, tacrine, TAK147, xaliproden, and mixtures thereof.

Suitable EGFR inhibitors include, but are not limited to gefitinib, erlotinib, lapatinib, canertinib, sorafenib, vandetanib, cetuximab, panitumumab, trastuzumab, pharmaceutical salts thereof and mixtures thereof.

The hydroxylamine derivatives may be provided to an individual by any suitable means, preferably directly (e.g., locally, as by injection to a nerve tissue locus) or systemically (e.g., parenterally or orally). Where the compound is to be provided parenterally, such as by intravenous, subcutaneous, intramuscular, intraorbital, ophthalmic, intraventricular, intracranial, intracapsular, intraspinal, intracisternal, intraperitoneal, buccal, rectal, vaginal, intranasal or by aerosol administration. According to a preferred embodiment, the pharmaceutical compositions of this invention are orally administered.

The amount of both the compound and the additional therapeutic agent that may be combined with the carrier materials to produce a single dosage form will vary depending upon the host treated and the particular mode of administration. Preferably, the compositions of this invention are formulated so that a dosage of between 0.1-1 g/kg body weight/day, preferably 0.1-300 mg/kg body weight, can be administered. The dose of the compound may depend on the condition and the illness of the patient, and the desired daily dose. In human therapy, the oral daily dose is preferably 10-300 mg. These doses are administered in unit dosage forms, which may be divided into 2-3 smaller doses for each day in certain cases, especially in oral treatment.

In the compositions of the present invention, the compounds may act synergistically in combination with each other and may further act synergistically in the presence of an additional therapeutic agent. Therefore, the amount of compound(s) and additional therapeutic agent(s) in such compositions may be less than that required in a monotherapy utilizing only that therapeutic agent. In such compositions a dosage of between 0.1-1 g/kg bodyweight/day of the additional therapeutic agent can be administered.

It should also be understood that a specific dosage and treatment regimen for any particular patient may depend upon a variety of factors, including the activity of the specific compound employed, the age, body weight, general health, sex, diet, time of administration, rate of excretion, drug combination, and the judgment of the treating physician and the severity of the particular disease being treated. The dosage of compound may also depend upon which particular compound is in the composition. Additionally, the effective amount may be based upon, among other things, the size of the compound, the biodegradability of the compound, the bioactivity of the compound and the bioavailability of the compound. If the compound does not degrade quickly, is bioavailable and highly active, a smaller amount will be required to be effective. The actual dosage suitable for a subject can easily be determined as a routine practice by one skilled in the art, for example a physician or a veterinarian given a general starting point.

The compound may be delivered hourly, daily, weekly, monthly, yearly (e.g., in a time release form) or as a one-time delivery. The delivery may be continuous delivery for a period of time, e.g., intravenous delivery. In one embodiment of the methods described herein, the therapeutic composition is administered at least once per day. In one embodiment, the therapeutic composition is administered daily. In one embodiment, the therapeutic composition is administered every other day. In one embodiment, the therapeutic composition is administered every 6 to 8 days, or more specifically, weekly.

In one embodiment, the therapeutic compounds or compositions described herein are administered in a sustained release form. Such method comprises implanting a sustained-release capsule or a coated implantable medical device so that a therapeutically effective dose of the hydroxylamine derivative is continuously delivered to a subject of such a method. The hydroxylamine derivative may be delivered via a capsule which allows sustained-release of the agent or the peptide over a period of time. Controlled or sustained-release compositions include formulation in lipophilic depots (e.g., fatty acids, waxes, oils). Also comprehended by the invention are particulate compositions coated with polymers (e.g., poloxamers or poloxamines).

Another aspect of the invention provides pharmaceutical compositions comprising a hydroxylamine derivative for the treatment of stroke, such as embolic stroke or thrombotic stroke or both. Such a composition comprises a hydroxylamine derivative and a pharmaceutically suitable carrier.

The materials are formulated to suit the desired route of administration. The formulation may comprise suitable excipients including pharmaceutically acceptable buffers, stabilizers, local anesthetics, and the like that are well known in the art. For parenteral administration, an exemplary formulation may be a sterile solution or suspension; for oral dosage, a syrup, tablet, capsule, gelcap, or palatable solution; for administration by inhalation, a microcrystalline powder or a solution suitable for nebulization; for intravaginal or intrarectal administration, pessaries, suppositories, creams or foams. A preferred formulation is a formulation for oral administration.

Suitable pharmaceutically acceptable carriers that may be used in these pharmaceutical compositions include, but are not limited to, ion exchangers, alumina, aluminum stearate, magnesium stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. In some embodiments, the pharmaceutically acceptable carrier is magnesium stearate. Additional pharmaceutical excipients commonly accepted and used are found in, for example, Remington's Pharmaceutical Sciences (Gennaro, A., ed.), Mack Pub., 1990, which is incorporated by reference herein.

As detailed below in the Examples, compounds useful in the present methods were administered to an animal model of ischemic stroke. The result showed that iroxanadine and especially arimoclomol accelerated the recovery of ischemic stroke without affecting the infarct size. This has been seen before with other compounds but to our knowledge never with an orally available drug. Arimoclomol worked as well or better than the best positive control, which is the injection of bFGF directly into the brain.

The following examples are intended to be illustrative. The examples are non-limiting, and the skilled artisan will recognize that other embodiments are contemplated by the present disclosure.

### EXAMPLES

### Example 1

### Functional Recovery Following Middle Cerebral Artery Occlusion (MCAO) in Rats

The purpose of this study was to evaluate the efficacy of arimoclomol and iroxanadine in enhancing neurological recovery in a model of permanent middle cerebral artery occlusion (MCAO) in rats. The permanent MCAO is a well accepted and considered to be a standard animal model for studying clinical aspects of stroke. (Stroke. 1999; 30:2752-2758.)

Forty male Sprague Dawley Rats, each weighing 300-400 g, which had been housed and handled for behavioral assessment for seven (7) days prior to surgery for acclimation purposes, were operated under anesthesia to create focal cerebral infarcts by permanent occlusion of the proximal right middle cerebral artery (MCA) according to modified Tamura model. Briefly, the rats were anesthetized with 2-3% halothane in the mixture of N₂O:O₂ (2:1), and were maintained with 1˜1.5% halothane in the mixture of N₂O:O₂ (2:1). The temporalis muscle was bisected and reflected through an incision made midway between the eye and the eardrum canal. The proximal MCA was exposed through a subtemporal craniectomy without removing the zygomatic arch and without transecting the facial nerve. The artery was then occluded by microbipolar coagulation from just proximal to the olfactory tract to the inferior cerebral vein, and was transected. Body temperature was maintained at 37.5° C.±0.5° C. throughout the entire procedure. Cefazolin (40 mg/kg; Baxter, Lot 06014.1, Exp. January 2009) was given i.p. one day before MCAO and just after MCAO to prevent infections.

The rats were randomly divided into 4 groups of 10 animals each. One group was given arimoclomol, p.o., starting at one hour after the occlusion at 200 mg/kg/d once daily for 3 days, then 50 mg/kg/d once daily for a total of 29 days. The second group was given iroxanadine using the same dosing regimen as for arimoclomol. The third group is a positive control group and is given intracistemally 1 μg (at 20 μg/ml, also containing 100 μg/ml of bovine serum albumin) beta-fibroblast growth factor (bFGF) one hour and one day (i.e. two administrations) after the occlusion. The fourth group is a negative control with administration of the vehicle only.

Animals were evaluated preoperation (day −1), then every 7 days after the operation (7, 14, 21, and 28 days) by forelimb and hindlimb placing tests and body swing test, which are widely accepted as standard tests indicative of functional recovery after a stroke model.

For the forelimb placing test, the examiner held the rat close to a tabletop and scored the rat's ability to place the forelimb on the tabletop in response to whisker, visual, tactile, or proprioceptive stimulation. Similarly, for the hindlimb placing test, the examiner assessed the rat's ability to place the hindlimb on the tabletop in response to tactile and proprioceptive stimulation. Separate sub-scores were obtained for each mode of sensory input (halfpoint designations possible), and added to give total scores (for the forelimb placing test: 0=normal, 12=maximally impaired; for the hindlimb placing test: 0=normal; 6=maximally impaired).

For the body swing test, the rat was held approximately one inch from the base of its tail. It was then elevated to an inch above a surface of a table. The rat was held in the vertical axis, defined as no more than 100 to either the left or the right side. A swing was recorded whenever the rat moved its head out of the vertical axis to either side. The rat must return to the vertical position for the next swing to be counted. Thirty (30) total swings were counted. A normal rat typically has an equal number of swings to either side. Following focal ischemia, the rat tends to swing to the contralateral (left) side.

The behavior was evaluated by giving the score ranging in the parentheses after each test:
Forelimb placing test (0-12), consisting of whisker tactile placing (0-2); visual placing (forward, sideways) (0-4); tactile placing (dorsal, lateral) (0-4); and proprioceptive placing (0-2);
Hindlimb placing test (0-6), consisting of tactile placing (dorsal, lateral) (0-4); and proprioceptive placing (0-2).

On day 28 after MCAO, rats in the control group and the group with the highest dosage are anesthetized deeply with Chloral Hydrate and perfused transcardially with normal saline (with heparin 2 unit/ml). Brains were removed and stored in 10% formalin. Fixed brains were then embedded with paraffin, and 5 μm coronal sections were cut using a microtome. Sections were then stained with hematoxylin and eosin (H&E). Seven sections (+4.7, +2.7, +0.7, −1.3, −3.3, −5.3 and −7.3, compared to bregma respectively) from each brain were photographed by a digital camera and the infarct area on each slice was determined by NIH Image (Image J) using the “indirect method” (area of the intact contralateral [left] hemisphere—area of intact regions of the ipsilateral [right] hemisphere) to correct for brain edema (Jiang et al., 1996). Infarct areas were then summed among slices and multiplied by slice thickness to give total infarct volume, which was expressed as a percentage of intact contralateral hemispheric volume.

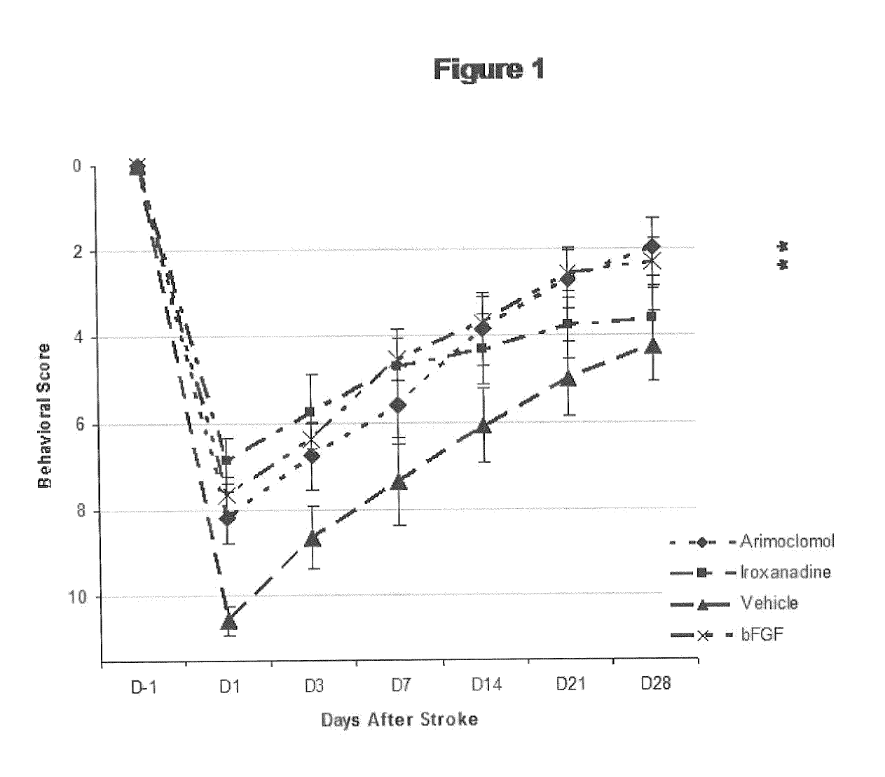

The experiments were carried out by the investigators who were blinded with regard to arimoclomol, iroxanadine and negative control. Because of the difference in dosing regimen, the bFGF group could not be blinded. All data are expressed as mean±S.E.M. Behavioral and body weight data were analyzed by repeated measures of ANOVA (treatment X time). Positive F-values for overall ANOVAs including all groups enabled pairwise ANOVAs between groups. Infarct volume data were analyzed by one-way ANOVA. In Figures below: *=different from vehicle-treated group by <0.05; **=different from vehicle-treated group by p<0.01; ***=different from vehicle-treated group by p<0.001. The results for arimoclomol are shown in FIGS. 1, 2, and 3.

FIG. 1 shows the results of the Forelimb Placing Test. Recovery in the bFGF group, the positive control, was superior to the vehicle group (p<0.01) as expected. Recovery in the arimoclomol group was superior to the vehicle group (p<0.05) at all time points. There was a non-significant trend of enhanced recovery in the iroxanadine group, compared to the vehicle group. The recovery scores at early time points (i.e. Days 1, 3, and 7) were superior to those of the vehicle group, indicating probable effectiveness of iroxanadine in the early stage of recovery from stroke, but the improvement slowed and was not sustained at time points farther from the occlusion event.

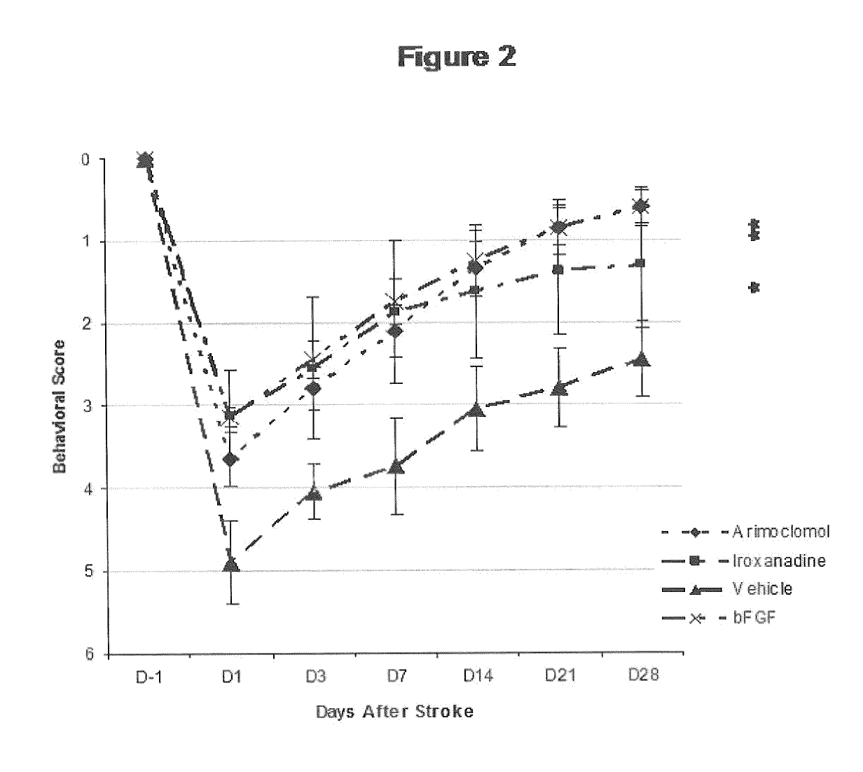

FIG. 2 shows the results of the Hindlimb Placing Test. Recovery in the bFGF group, the positive control, was superior to the vehicle group (p<0.001). Similar to the results of the Forelimb Placing Test, recovery in the arimoclomol group was superior to the vehicle group (p<0.001) at all time points, indicating continued effectiveness of arimoclomol to enhance recovery from stroke. Recovery in the iroxanadine group was superior to the vehicle group (p<0.01). The recovery scores for iroxanadine group continued to improve, but at a slower rate than for the arimoclomol or the positive control group as more time passed after the occlusion event.

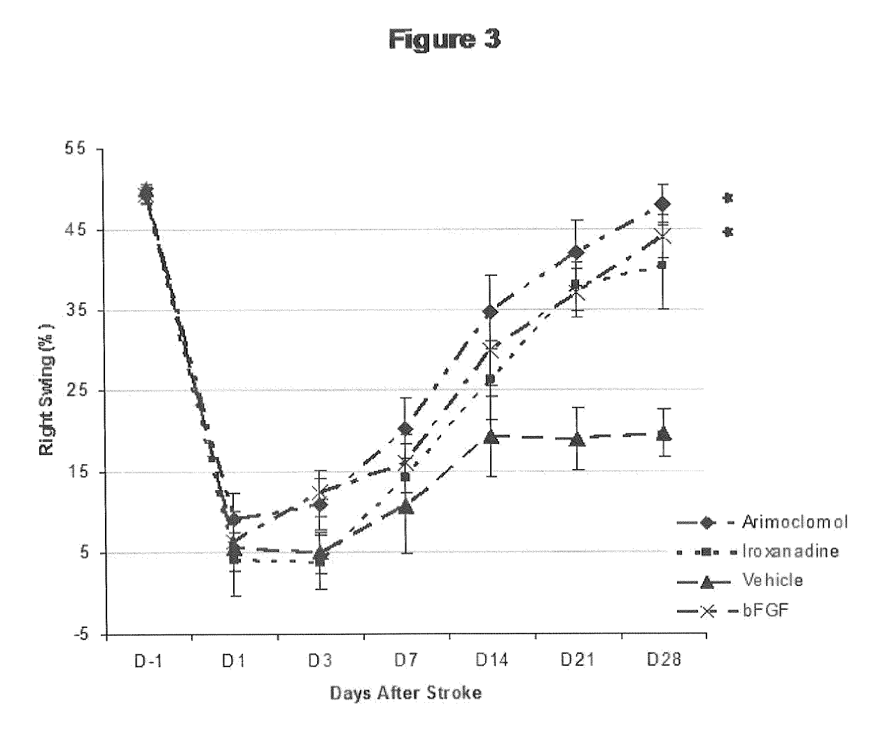

FIG. 3 shows the results of the Body Swing Test. Recovery in the bFGF group was superior to the vehicle group (p<0.05). Recovery in the arimoclomol group was superior to the vehicle group (p<0.01). There was a non-significant trend of enhanced recovery in the arimoclomol group compared to the vehicle group.

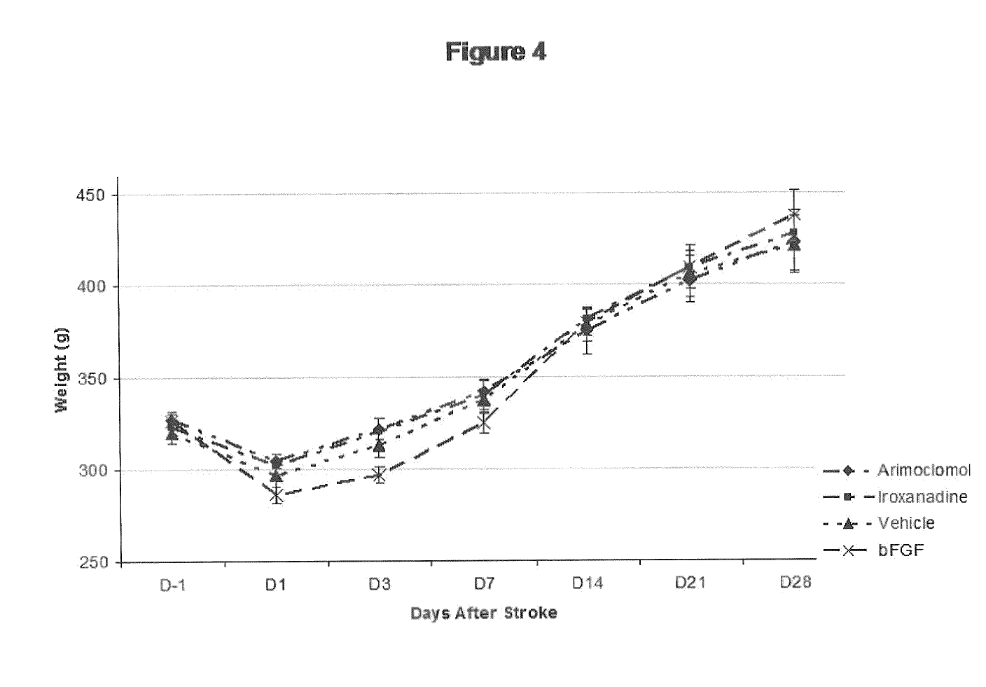

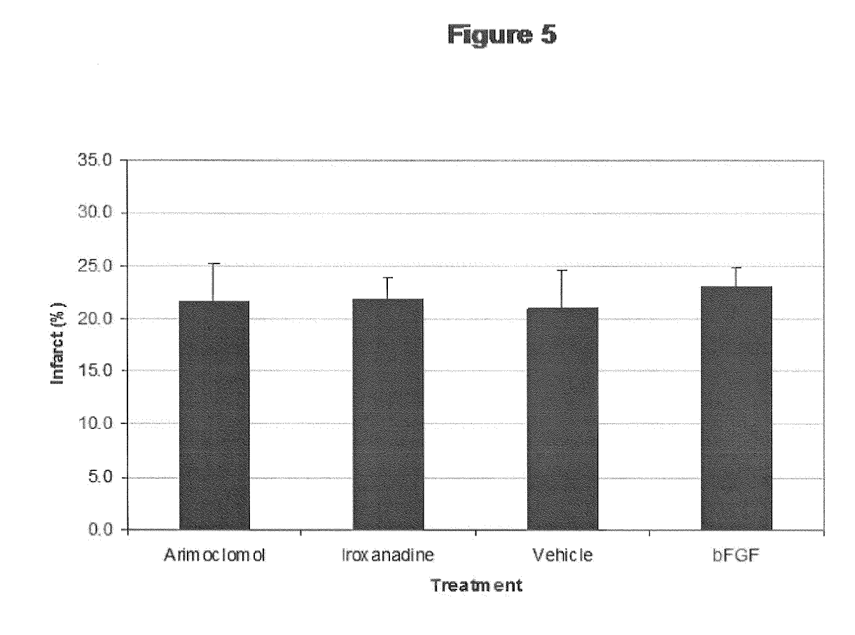

FIG. 4 shows that there was no significant difference in body weight among different groups, and FIG. 5 shows that the infarct size has not changed.

There was no significant changes in the body weight of the test animals, and there was no significant changes in the infarct volumes of the test animal brains. See Table I.

TABLE I
Infarct Volume (%)

| | |
|---|---|
| arimoclomol | 21.6 ± 3.6 |
| iroxanadine | 21.8 ± 2.1 |
| Vehicle | 21.0 ± 3.5 |
| bFGF | 23.1 ± 1.7 |

In conclusion, significant differences were seen in neurological recovery among the four groups. In all behavioral tests (the forelimb placing test, the hindlimb placing test and the body swing test), the arimoclomol group and the bFGF group showed significant enhancements of recovery compared to vehicle. The iroxanadine treated group showed significant enhancement of recovery in hindlimb placing and a trend toward enhancement of recovery in forelimb placing and body swing tests. There were no significant differences in body weight or infarct volume among groups. These results show enhancement of sensorimotor recovery in arimoclomol- and iroxanadine-treated animals. The forelimb and hindlimb placing tests reflect recovery of function of the sensorimotor cortex, whereas the body swing test reflects recovery of function of the striatum. Overall, these results indicate that arimoclomol and iroxanadine, and compounds sharing common structural elements and compounds that are structurally related to and functionally equivalent with either of these hydroxylamine derivatives, are useful as drugs that enhance sensorimotor functional recovery after stroke. The time course of recovery indicates that arimoclomol, which showed improved recovery score at all test time points, and iroxanadine, which tended to enhance recovery at earlier test time points, may each exert its effects at a different time point after the occlusion event. Therefore, it is probable that the combination therapy using these two compounds may have an additive or synergistic effect, with iroxanadine assisting recovery at earlier time points, e.g. up to 7 days after the occlusion, and arimoclomol providing a continuous improvement beyond 7 days, up to 28 days after the occlusion or later.

### Example 2

### Functional Recovery Following MCA Occlusion in Rats with Administration of Arimoclomol—Dose Study

Fifty male Sprague Dawley Rats, each weighing 300-400 g, are operated under anesthesia to create MCA occlusion as described in Example 1, and are divided into 5 groups of 10 animals each. Each group is given arimocolomol, p.o., starting at one day after the occlusion at 25 mg/kg/d, 50 mg/kg/d, 100 mg/kg/d, or 200 mg/kg/d once daily for 35 days. One group is a control group with administration of the vehicle only.

Animals are evaluated pre-operation (day −1), then every 7 days after the operation (7, 14, 21, 28, and 35) by forelimb and hindlimb placing tests and body swing test, and given scores as described in Example 1.

On day 35 after MCAO, rats in the control group and the group with the highest dosage were sacrificed and their brains evaluated as in Example 1. The rats in other groups are sacrificed and the brains were removed and flash frozen for further analysis.

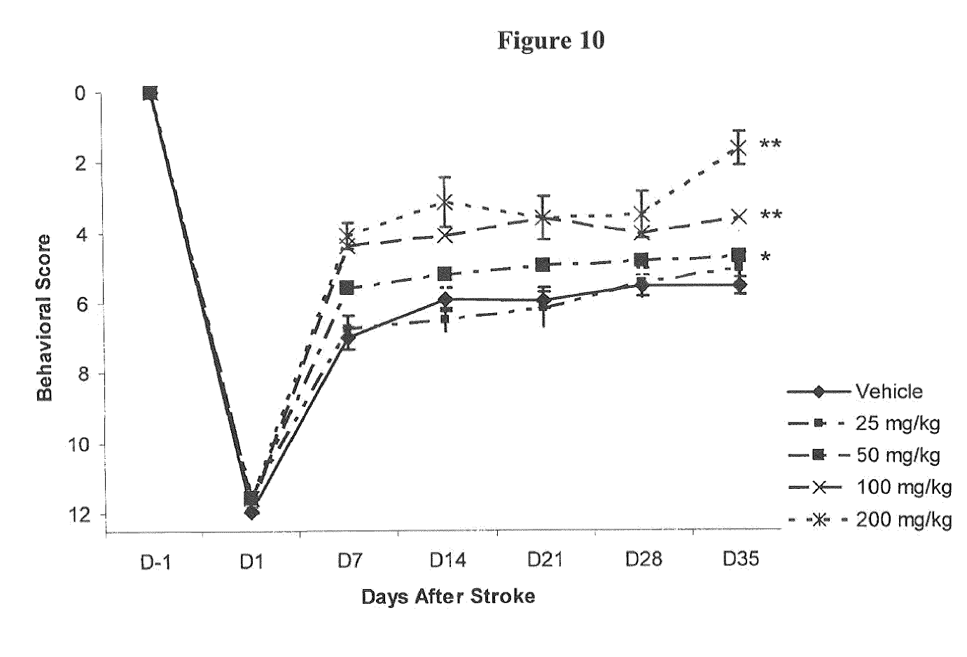

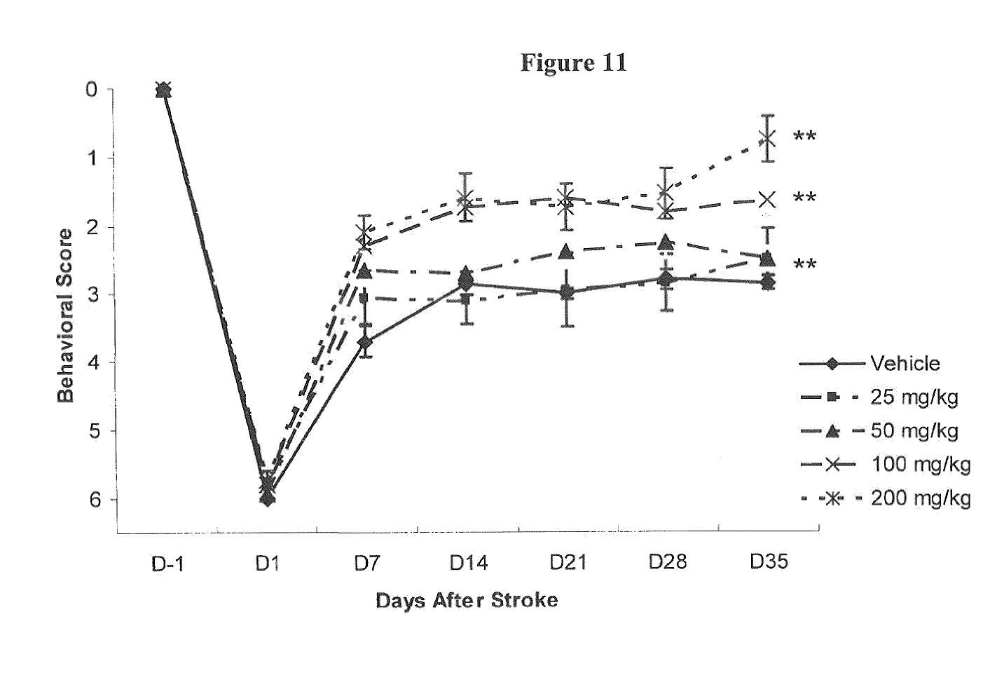

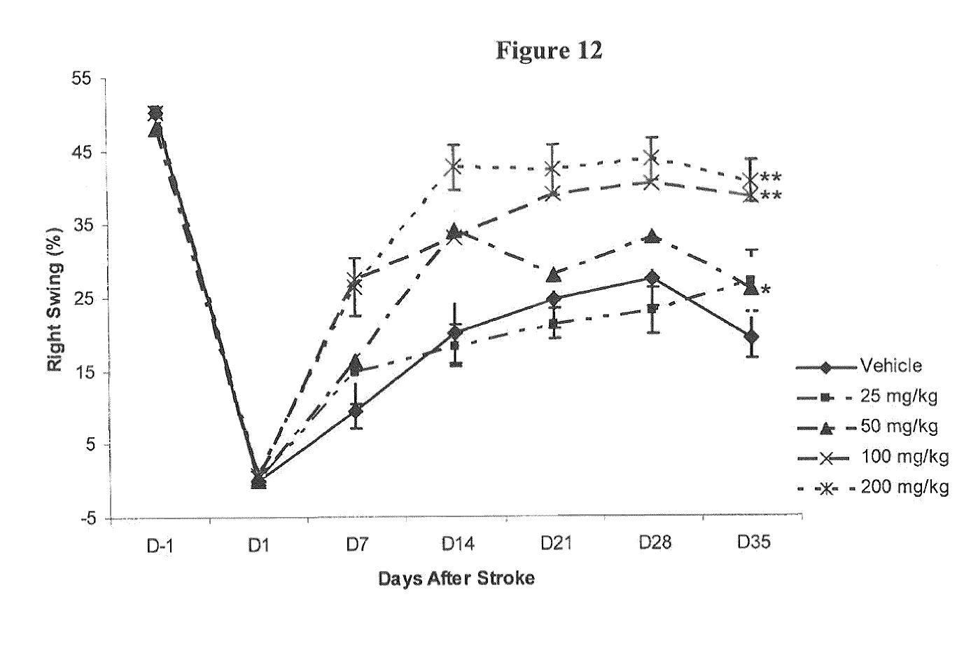

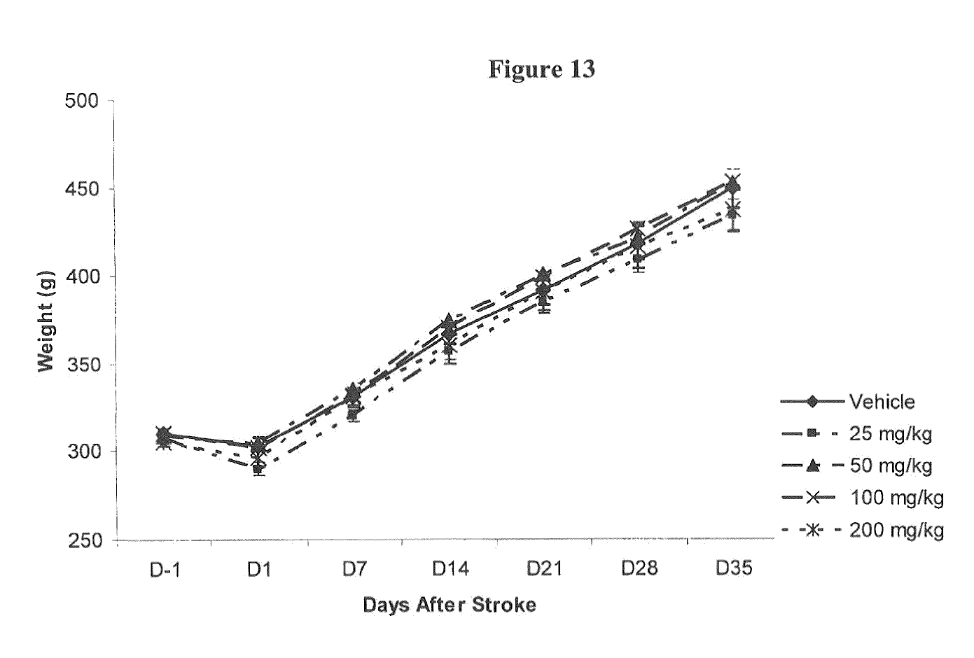

The results for the forelimb placement, hindlimb placement and body swing tests shown in FIGS. 10, 11 and 12 illustrate that there was significant dose related improvement of the behavioral scores starting at 50, 100 and 200 mg/kg/day doses of arimoclomol. There was no significant difference shown between the vehicle control group and the 25 mg/kg/day dose group. The results of these three tests independently confirmed results in Examples 1 and 3 indicating that arimoclomol is effective in the stroke recovery model at doses ranging from 50-200 mg/kg/day, with 200 mg/kg/day as the maximum effective dose in the study. In addition, no changes in body weight or infarct volume were observed. This was similar to the results in Examples 1 and 4.

### Example 3

### Functional Recovery Following MCA Occlusion in Rats with Administration of Iroxanadine

### Dose Study

The experiment of Example 2 is also carried out using iroxanadine as the therapeutic agent, except using a higher dosage amount of 50 mg/kg/d, 100 mg/kg/d, 200 mg/kg/d, or 400 mg/kg/d once daily for 35 days. The results of Example 1 showed that there was a non-significant trend of improved recovery in animals administered iroxanadine under the given dosage. This example is expected to more clearly indicate the efficacy of iroxanadine in enhancing recovery from stroke.

### Example 4

### Functional Recovery Following MCA Occlusion in Rats with the Administration of Arimoclomol

### Therapeutic Window

Fifty male Sprague Dawley Rats, each weighing 300-400 g, are operated under anesthesia to create MCA occlusion as described in Example 1, and are divided into 5 groups of 10 animals each. Each group is given arimoclomol, p.o., starting at 6, 12, 24, and 48 hours after the occlusion at 200 mg/kg/d once daily for 35 days. One group is a control group with administration of the vehicle only at 6 hours. The animals are evaluated as described in Example 2.

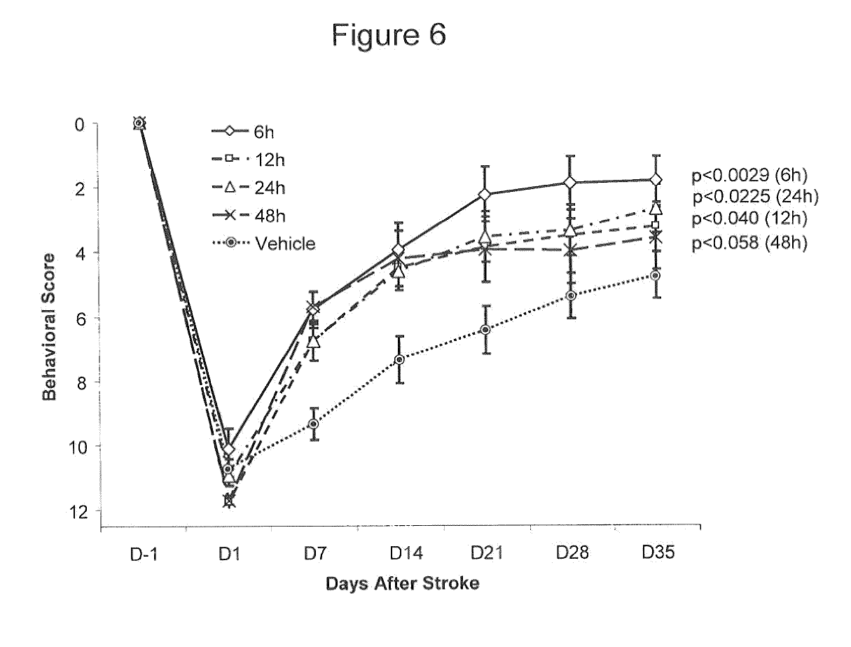

FIG. 6 shows the results of the Forelimb Placing Test after the administration of arimoclomol to rats. Consistent with the results of the experiment described in Example 1, recovery in all groups that received arimoclomol at various time points after the occlusion was superior to the vehicle group. The results over the time course of recovery for each of the treatment groups were superior to the vehicle group (ANOVA, p<0.06). Recovery was significant and sustained even when arimoclomol was administered for the first time 48 hours after occlusion; however, in these experiments under these conditions, recovery was superior in the group that received arimoclomol 6 hours after stroke. This result suggests that the therapeutic mechanism of arimoclomol may operate at both early and late time in the stroke recovery process.

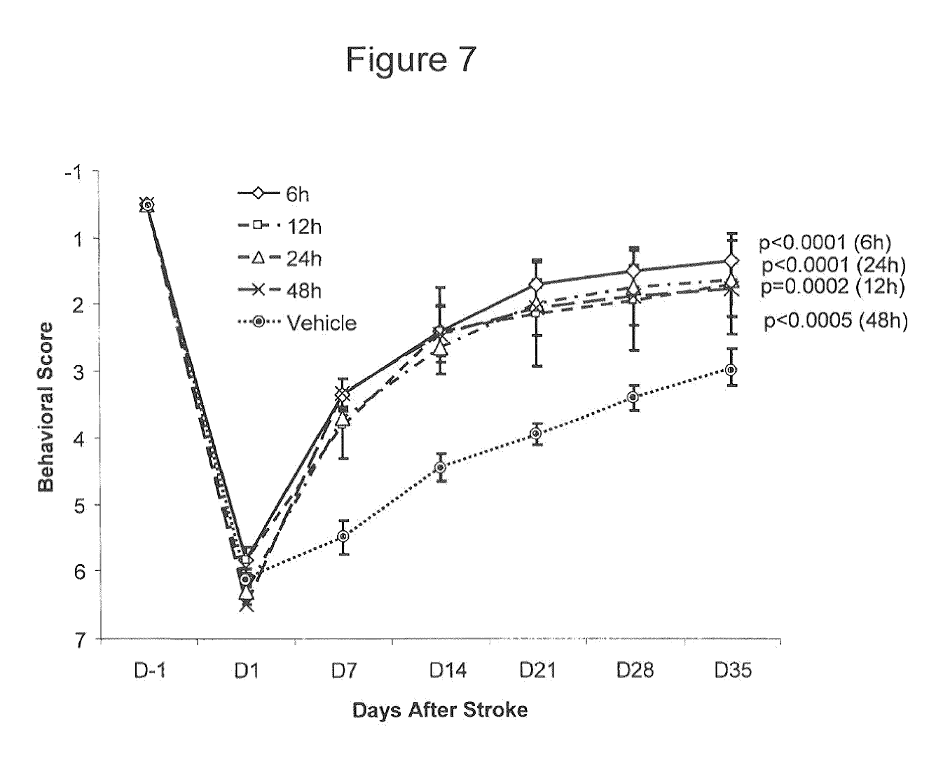

FIG. 7 shows the results of the Hindlimb Placing Test after the administration of arimoclomol. Similar to the results of the Forelimb Placing Test, the time course of recovery in each arimoclomol-treated groups was superior to the vehicle group (ANOVA, p<0.001), indicating continued effectiveness of arimoclomol to enhance recovery from stroke. There was no statistical difference among treated groups, regardless of whether the first administration of arimoclomol was 6, 12, 24, or 48 hours after occlusion, indicating that arimoclomol is highly effective in promoting recovery, even when administered after long periods of time, at least up to 48 hours, from occlusion.

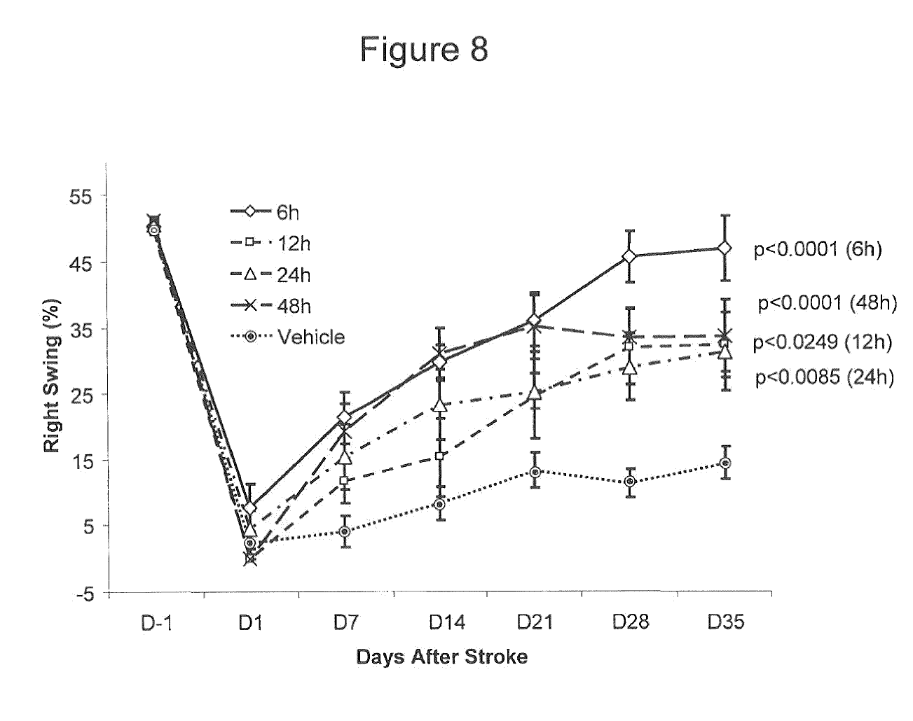

FIG. 8 shows the results of the Body Swing Test after the administration of arimoclomol. Recovery in all arimoclomol groups was superior to the vehicle group (ANOVA, p<0.03) over the recovery time course. Recovery of the group that received arimoclomol 48 hours after occlusion was indistinguishable from those that received arimoclomol 6 hours after occlusion for time points up to and including 21 days after occlusion. In these experiments, under these conditions, the group that received arimoclomol 6 hours after occlusion continued to show improved performance after 21 days, whereas other groups tapered off. This result suggests that the therapeutic mechanism of arimoclomol may operate at both early and late time in the stroke recovery process and that early effects of arimoclomol may continue to have positive effects even late in the recovery process.

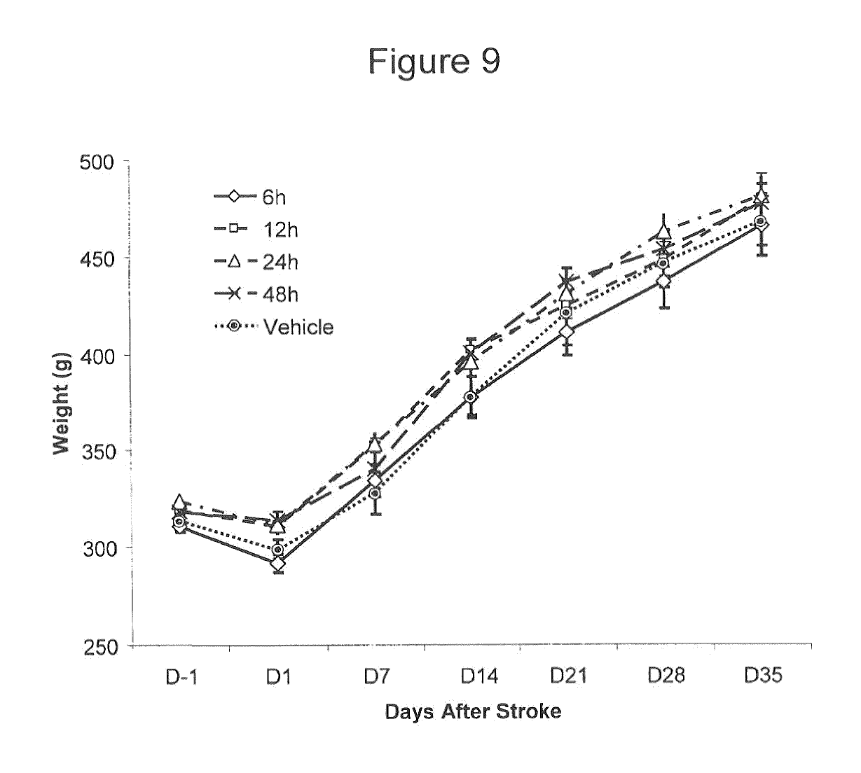

FIG. 9 shows that there was no significant difference in body weight among different groups.

### Example 5

### Functional Recovery Following MCA Occlusion in Rats with Administration of Iroxanadine

### Time Course

The experiment of Example 4 is also carried out using iroxanadine as the therapeutic agent. The results are expected to show the efficacy of iroxanadine at a time later than currently available therapeutic agents.

### Example 6

### Functional Recovery Following MCA Occlusion in Rats with Administration of a Combination of Arimoclomol and Iroxanadine

The experiments described in Examples 2-5 are carried out administering both iroxanadine and arimoclomol. The results are expected to show no interference of the compounds with each other, and at least additive and perhaps synergistic effect of the two therapeutic compounds.

### Example 7

### Neuroprotection from Damages from Transient Global Cerebral Ischemia in Gerbils with Administration of Compound 2

Adult male Mongolian gerbils weighing 60-80 g were intraperitoneally administered 50 mg/kg of Compound 2 prior to surgery to induce transient global cerebral ischemia. The gerbils were operated under anesthesia with 2% halthane in a gas mixture of 70% N₂O and 30% O₂, followed by maintenance at 1% halothane to surgically expose and clamp the carotid arteries for 5 minutes to prevent the blood flow to the brain. After 5 minutes, the clamps were removed and reperfusion occurred. The gerbils were evaluated for motility and spontaneous alteration in a Y maze test essentially as described in Gupta and Sharma, Biol. Pharm. Bull. 29(5) 957-961 (2006), and were decapitated 4 days after reperfusion for histological evaluation for the neuronal survival in the hippocampal CA-1 area.

In two experiments, Compound 2 showed neuroprotective effects, allowing the gerbils in the test group to show improved outcomes in comparison to the controls group, which were given only the carrier.

### Example 8

### Protective Effect of Compound 2 in Rats undergoing Experimental Cardiac Ischemia/Reperfusion

Rats experienced significantly decreased duration of ventricular arrhythmias and increased overall survival when Compound 2 was administered prior to a standard cardiac ischemia/reperfusion model known to one skilled in the art.

### Example 9

### Protective Effect of Compound 2 in Mice under Cytotoxic Hypoxia

Mice that were administered Compound 2 had a significantly increased survival time under conditions of stress (cytotoxic hypoxia) induced by hydrogen cyanide poisoning. The cytotoxic hypoxia was induced by a standard protocol. Compound 2 was not overtly toxic, having a LD50 of about 1000 mg/kg in mice. Separately, Compound 2 was tested and was found not to be mutagenic in standard bacterial reverse mutation assays.

### Example 10

### Rescue of Rat Cortical Neurons After Oxygen/Glucose Deprivation

10.1 Cell Culture

Mixed cortical cultures were prepared from E18 Wistar rat embryos (National Animal Center, Kuopio, Finland). The cortices were dissected and the tissue cut into small pieces. The cells were separated by fifteen minute incubation with DNase and papain. The cells were collected by centrifugation (1500 rpm, 5 min). The tissue was triturated with a pipette and the cells were placed on poly-L-lysine coated 48-well plates, 300,000 cells/cm2, in MEM (2 g/L glucose) supplemented with 2 mM glutamine, 0.1 μg/mL gentamicin, 10% heat-inactivated fetal bovine serum (FSB-HI) and 10% heat-inactivated horse serum (HS-HI). After 3-4 hours, the medium was changed to MEM (2 g/L glucose) and supplemented with 2 mM glutamine, 0.1 μg/mL gentamicin, 5% HS-HI. After three days in vitro, cells were transferred into medium containing MEM (2 g/L glucose) supplemented with glutamine, gentamicin and 5% each of FSB-HI and HS-HI sera. On day 6 in vitro, unwanted cell division was inhibited by adding cytosine arabinoside (10 μM final concentration) for 24 hours. The cultures were transferred into MEM (2 g/L glucose) supplemented with glutamine, gentamicin and 5% HS-HI before further experiments were performed.

0.2 Oxygen Glucose Deprivation (OGD)

Wells containing cells with a healthy appearance were chosen for experiments on day 10 in vitro. Arimoclomol was diluted in balanced salt solution. As a control for total neuronal death, 300 μM NMDA was used for 24 hours, and oxygen and glucose were removed from the cultures for 5 hours to induce approximately 30-60% cell death. Wells treated with medium only and incubated under normoxic conditions served as the zero control. One hour after the initiation of OGD, arimoclomol was added to the wells at 0.1, 0.2, 0.5, 1, 2, 5, 10, 20, and 50 μM concentration. The cells were flushed again and after 4 hours, the medium was removed, normal culture media containing the compounds were pipetted into the wells and the plates were placed into a normoxic incubator for an additional 20 hours.

10.3 Assessment of Cell Death by LDH Release

After 24 hours, possible cell debris was removed by centrifugation (13,000 rpm, 3 min.). Two 100 mL aliquots of culture medium were pipetted into a microtiter plate, and an equal amount of LDH reagent was pipetted into the wells. The absorbance at 340 nm was measured immediately using a 3 min. kinetic measurement protocol in Multiskan ELISA reader (Labsystems, Finland). The change in absorbance/min was determined, which is directly proportional to the released LDH (=cell death).

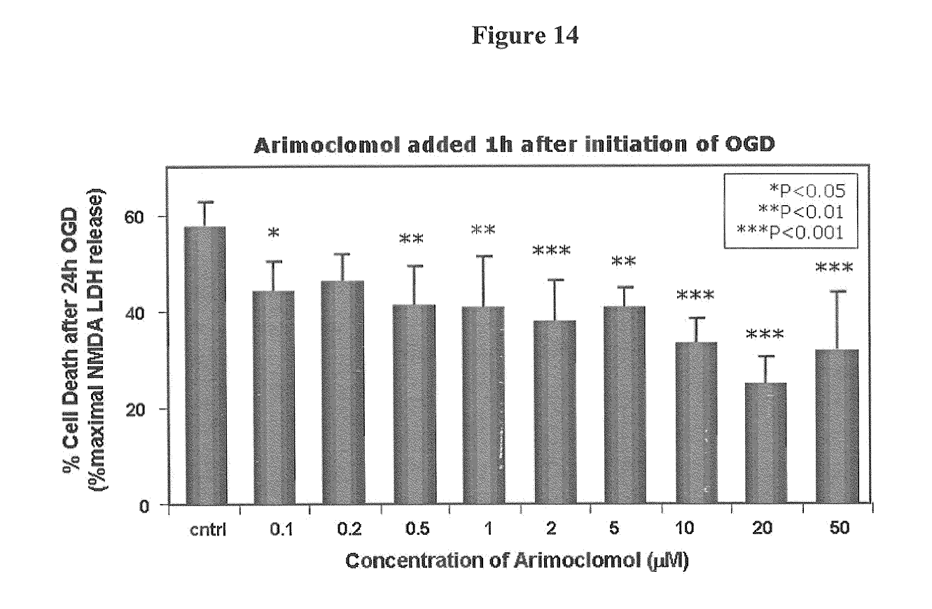

FIG. 14 shows that in Oxygen Glucose Deprivation (OGD), all concentrations of arimoclomol except 0.2 μM were cytoprotective against OGD when administered to the cells one hour after the onset of oxygen and glucose deprivation, as measured by LDH release.

### Example 11

### Pharmacokinetic Data in Rats

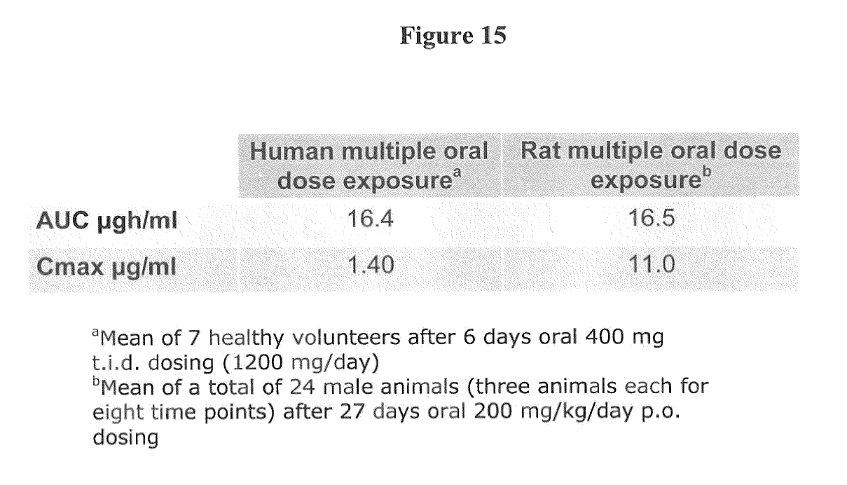

The pharmacokinetic study included one control group and three dosed groups (200 mg/kg/day, 400 mg/kg/day, and 900 mg/kg/day) for six months. The control and three dose groups (n=96 animals/sex/group) were treated with the vehicle and with appropriate concentrations of arimoclomol orally by gavage for six months. Blood samples were harvested from three animals at 8 time points—0 (pre-dose), 0.5, 1, 2, 4, 8, 12, and 24 hours after dosing—on days 0, 27, 89, and 180 for males and on 0, 27, 89, and 181 for females. The serum concentration of arimoclomol was measured by HPLC. The pharmacokinetic parameters (AUC and Cₘₐₓ) were estimated using the WinNonlin v5.0.1 pharmacokinetic software. FIG. 15 shows the mean AUC and Cmax values after 27 days oral 200 mg/kg/day dosing.

### Example 12

### Pharmacokinetic Clinical Study in Humans

Seven healthy human volunteers were administered orally 400 mg t.i.d. dosing (1200 mg/day) in a double-blinded and placebo controlled clinical study. No clinical abnormalities or changes were observed for ECGs, vital signs, clinical chemistry or physical examination, including body weight. FIG. 15 shows that the level of exposure in the 200 mg/kg/day rat PK study can be safely achieved in human volunteers.

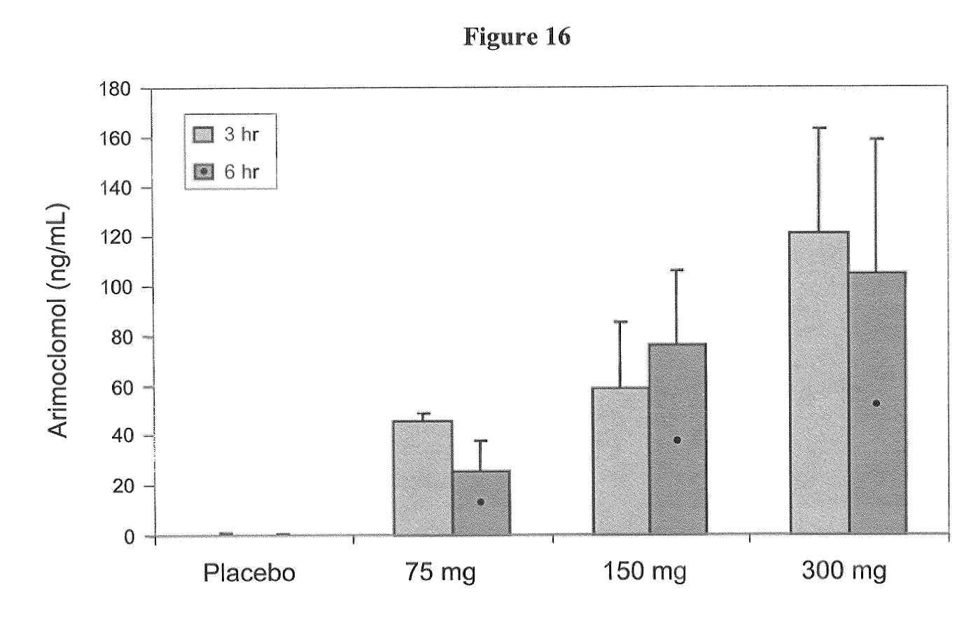

FIG. 16 shows that arimoclomol penetrates the human blood-brain barrier by measuring cerebrospinal fluid (CSF) levels at 3 and 6 hours after oral administration with increasing doses of arimoclomol.

### Example 13

### Functional Recovery Following Embolic Middle Cerebral Artery Occlusion (MCAO) in Rats

The purpose of this study was to evaluate the efficacy of arimoclomol in enhancing neurological recovery in a model of embolic middle cerebral artery occlusion (MCAO) in rats (Zhang R L et al. Brain Research 766:83-92, 1997). The embolic MCAO model emulates the damage caused by an embolic stroke which is generally the result of ischemia associated damage or diseases. The embolic MCAO model also closely models the types of damage caused by a stroke because the occlusion is not permanent. Moreover, the embolus often undergoes spontaneous clot lysis and reperfusion within 24-48 hours post-stroke, which is similar to a subset of human stroke. Damage from a stroke can be brought about in two ways. First, the original occlusion and cut off of blood supply can significantly damage neurological behavior and the effected blood vessels. Second, the resulting pressure from the initial surge in blood volume once the occlusion is cleared can also result in further neurological damage and damage to the effected blood vessel. This embolic stroke model provides guidance for the neurological recovery resulting from each of these phenomena.

Sixty male Wistar rats between 8-12 weeks of age were operated on to isolate the external carotid artery (ECA) and the internal carotid artery (ICA). A single clot was introduced into the ECA lumen through a PE-50 catheter (0.3 mm outer diameter) attached to a 100 μL Hamilton syringe filled with 0.9% saline via a small puncture. A 16 mm length of the PE-50 catheter was advanced from the ECA into the lumen of the ICA. Subsequently, the clot was injected into the ICA with 2-3 μL of 0.9% saline. The catheter was withdrawn and the right ECA ligated.

The rats were then randomly divided into 6 groups of 10 animals each. To the control group, phosphate buffered saline (PBS) was administered, p.o., starting at six hours after embolic stroke for 28 consecutive days. To the second group, Erythropoitein (EPO) was administered, i.p., starting at 24 hours after embolic stroke for 7 consecutive days (41.7 μg/kg/d). To the third group, arimoclomol was administered daily p.o. (200 mg/kg/d), starting 6 hours after embolic stroke for 28 consecutive days. To the fourth group, arimoclomol was administered daily p.o. (200 mg/kg/d), starting 10 hours after embolic stroke for 28 consecutive days. To the fifth group, arimoclomol was administered p.o. (200 mg/kg/d), starting 24 hours after embolic stroke for 28 consecutive days. To the sixth group, arimoclomol was administered p.o. (200 mg/kg/d), starting 48 hours after embolic stroke for 28 consecutive days.

Animals were evaluated at 1, 3, 7, 14, 21, and 28 days after initiation of embolic stroke by a modified neurological severity score (mNSS) and adhesive-removal and foot fault tests. These tests are widely accepted as standard tests indicative of functional recovery after a stroke. (Chen et al., Ann Neurol 53: 743-751, 2003; Wang et al., Stroke 35: 1732-1737, 2004; Zhang et al, Circulation 3486-3494, 2005).

For the modified neurological severity score (mNSS), the severity of injury was graded on a scale of 0 to 18. This score was determined via the evaluation of a variety of motor tests, sensory tests, balance beam tests and reflex tests as illustrated in Table II below. To determine the score resulting from these tests, one point was awarded for the inability to perform any of the tests or for the lack of a tested reflex. Accordingly, the higher the score, the more severe the injury. (Chen et al., Ann Neurol 53: 743-751, 2003; Wang et al., Stroke 35: 1732-1737, 2004; Zhang et al, Circulation 3486-3494, 2005).

TABLE II
Modified Neurological Severity Score Tests Summary

| | Possible |
|---|---|
| Type of Tests: | Points |
| Motor tests | 3 |
| A. Raising the rat by the tail: | |
| Flexion of forelimb 1 point | |
| Flexion of hindlimb 1 point | |
| Head moved more than 10o vertically within 30 secs. 1 point | |
| B. Walking on the floor (normal = 0 points; maximum = 3 points): | |
| Normal walk 0 points | |
| Inability to walk straight 1 point | |
| Circling toward the paretic side 2 points | |
| Fall down to the paretic side 3 points | |
| Sensory tests: | 2 |
| Placing test (visual and tactile test) 1 point | |
| Proprioceptive test (Pushing the paw to stimulate limb muscles) 1 point | |
| Balance Beam Tests (normal = 0 points; maximum = 6 points): | 6 |
| Balances with steady posture 0 points | |
| Grasps side of beam 1 point | |
| Hugs the beam and one limb fall down from the beam 2 points | |
| Two limbs fall down from the beam, or spins on beam (>60 sec) 3 points | |
| Attempts to balance on the beam but fall off (>40 sec) 4 points | |
| Attempts to balance on the beam but fall off (>20 sec) 5 points | |
| Fall off: No attempt to balance or hang on to the beam (<20 sec) 6 points | |
| Reflexes and Abnormal Movements Tests: | 4 |
| Pinna reflex (a head shake when touching auditory meatus) 1 point | |
| Corneal reflex (an eye blink when lightly touching cornea with cotton) 1 point | |
| Startle reflex (a motor response to a brief noise) 1 point | |
| Seizures, myoclonus, myodystony 1 point | |
| Maximum points: | 18 |

For the adhesive removal test, the rats are trained prior to embolic stroke to remove strips of packing tape (5 cm). The training period constitutes of 3 sessions. Each session consists of 6 bilateral stimulation trials conducted over 2 days wherein the 5 cm strips of packing tape were placed firmly around both wrists of the animal so that they covered the hairless part of the forepaw. The latency to contact the paws and the latency to remove the adhesive tape strip in both the ipsilateral and contralateral sides to the infarction were measured.

The foot fault test was carried out according to the protocol of Hernandez et al. (Exp. Neuro., 102:318-324, (1988)). The number of faults for the forepaw contralateral to the infarction was recorded along with the number of successful steps and displayed as a percentage of contralateral forelimb foot faults per forelimb steps.

On day 28 after embolic stroke, all animals were anesthetized deeply with Chloral Hydrate and perfused transcardially with heparinized saline followed by 4% paraformaldehyde. Brains were removed and stored in 4% paraformaldehyde. Infarct volume was then measured on seven hematoxylin and eosin (H&E) stained coronal sections using a Global Lab Image analysis program. The area of both hemispheres and the infarct area (mm²) was calculated by tracing the area on the display screen. Infarct volume (mm³) was determined by multiplying the appropriate area by the section interval thickness. The infarct volume was then calculated as a percentage of infarct volume of the contralateral hemisphere.

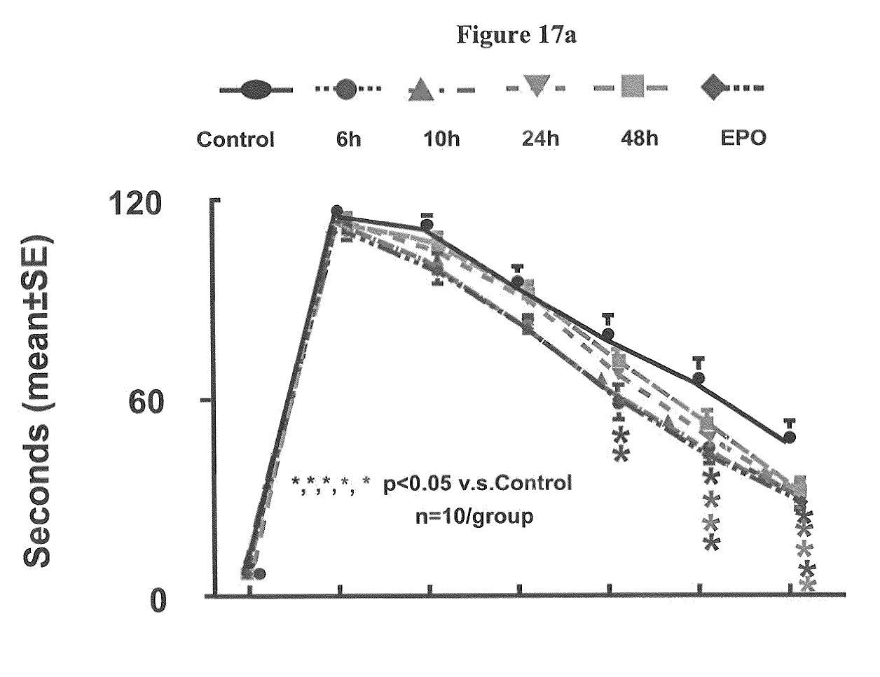

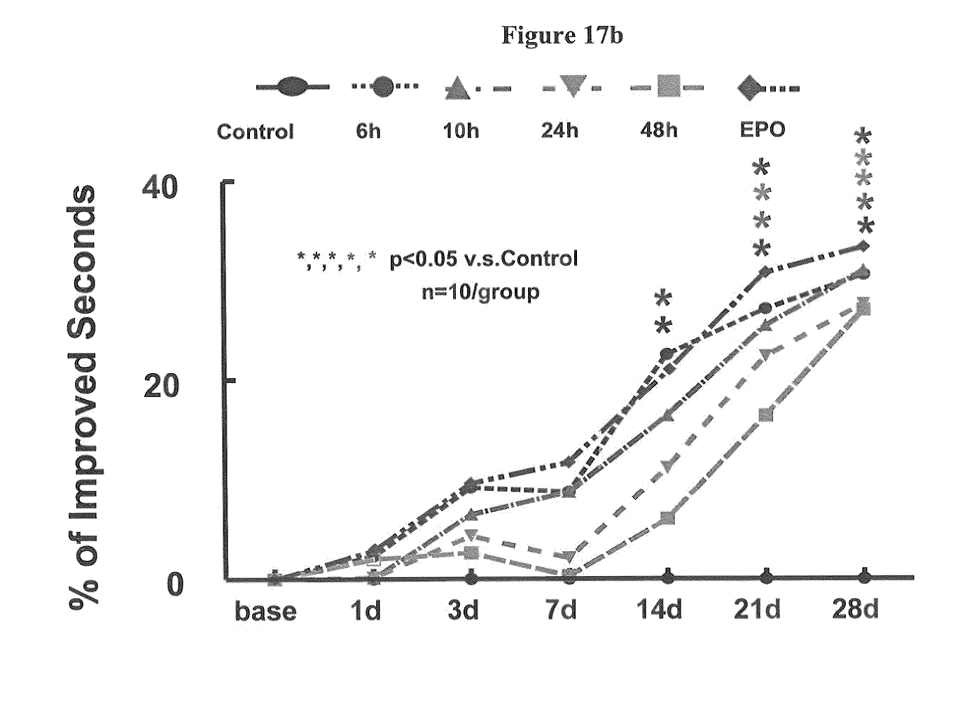

FIGS. 17a-b show the results of the adhesive-removal test. In FIG. 17a, recovery in each of the arimoclomol treatment groups (6 h, 10 h, 24 h and 48 h) was significantly superior to each of the corresponding control group treatments after the 14 day time point. FIG. 17b illustrates the percent improvement over the corresponding control at each time point (i.e., control is 0% for each time point). At day 28, treatment with arimoclomol in each time group showed an improvement percentage greater than 25% when compared to the corresponding control treatment group.

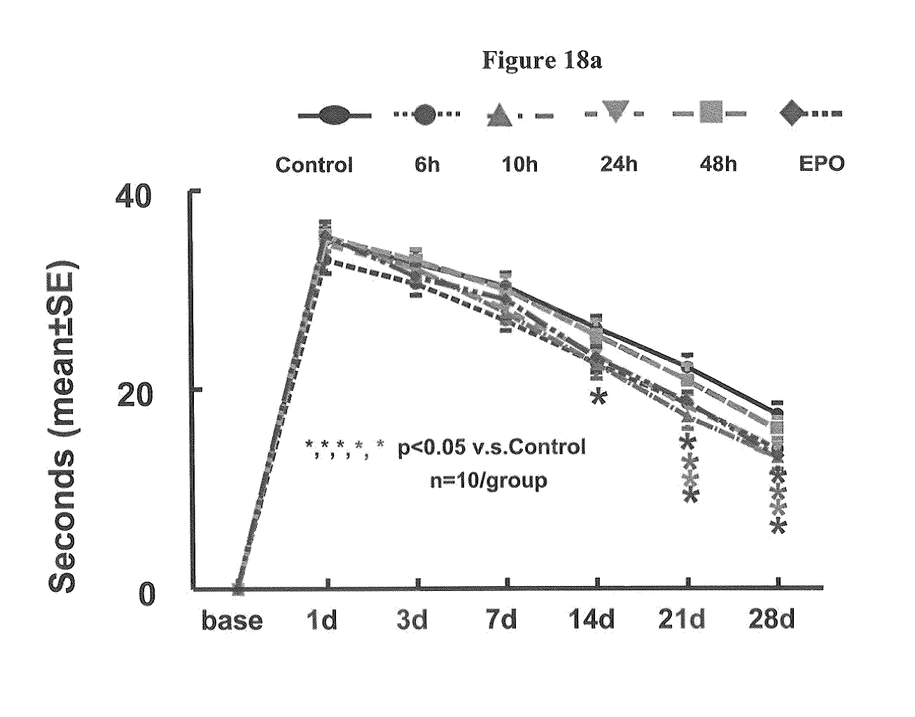

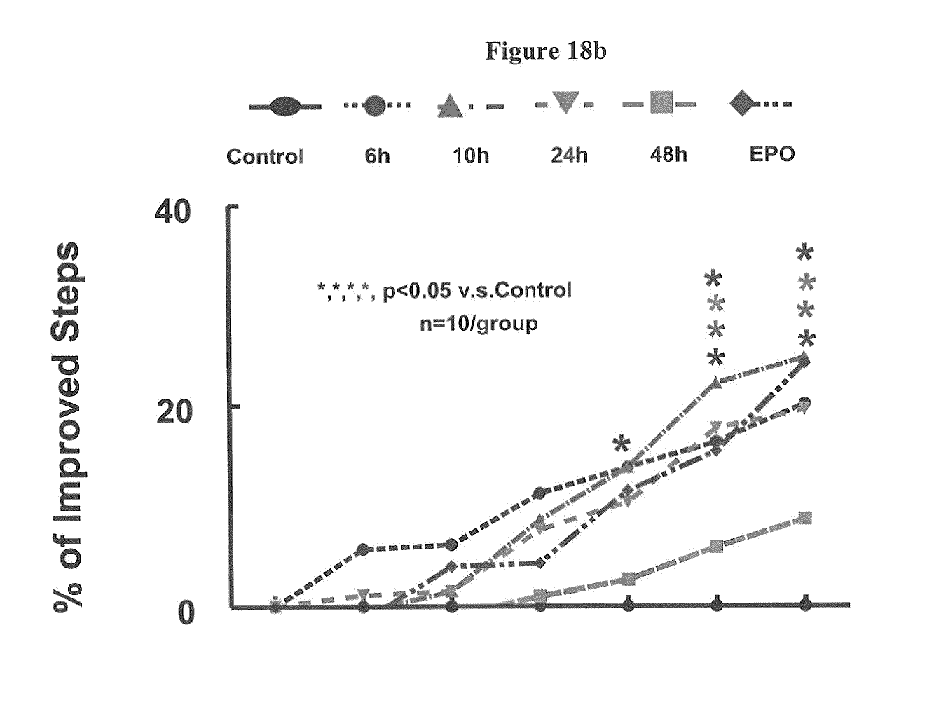

FIGS. 18a-b show the results of the foot fault test. FIG. 18a illustrates that recovery in the 6 h, 10 h and 24 h arimoclomol treatment groups were significantly superior to the corresponding control groups on and after the 14-day time point. FIG. 18b illustrates similar recovery as in FIG. 18a and also illustrates that the 10 h arimoclomol treatment group was significantly superior to the EPO treatment group at the 21-day time point and slightly superior at the 28-day time point.

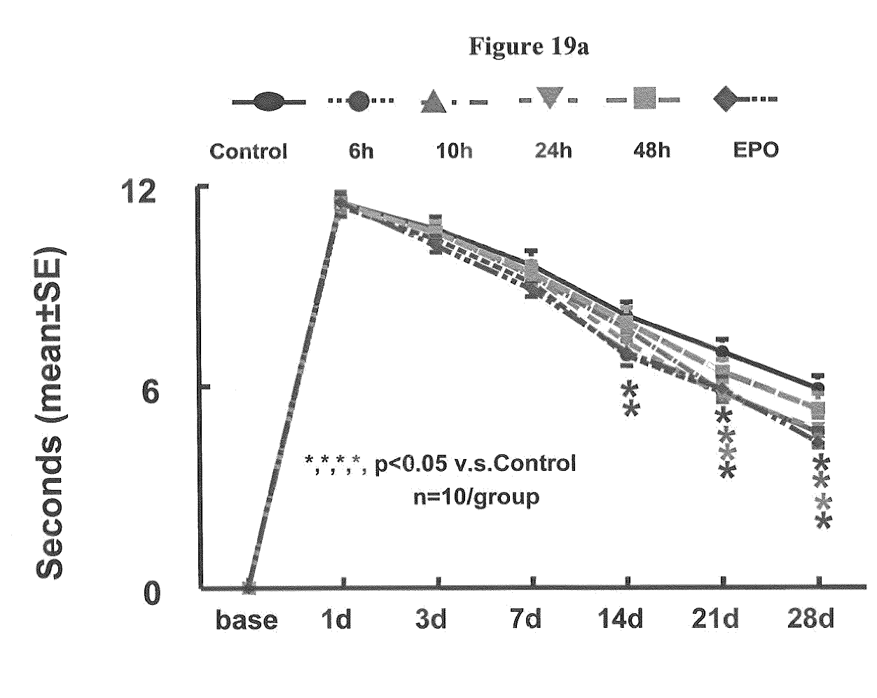

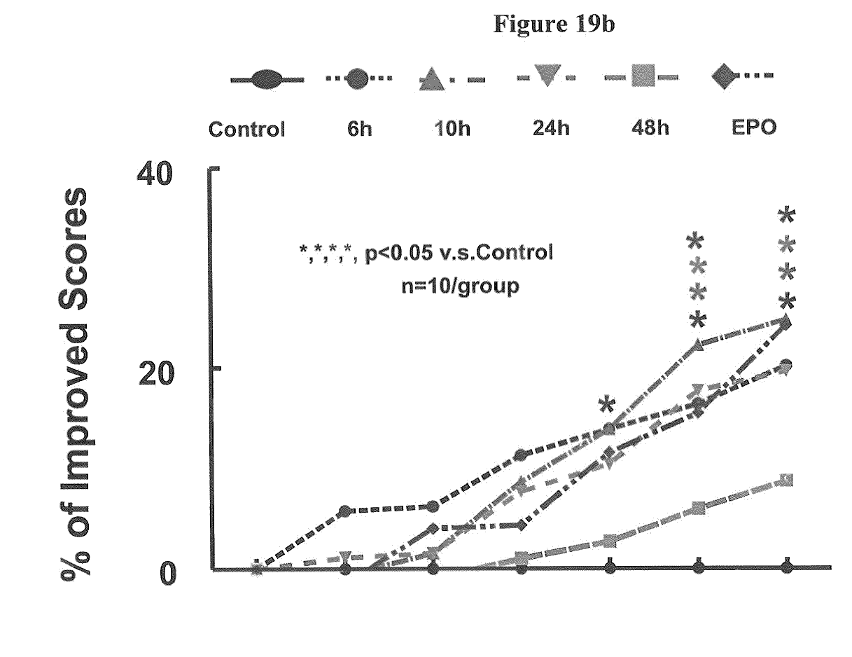

FIGS. 19a-b show the results of the mNSS test. FIG. 19a illustrates that recovery in the 6 h, 10 h, and 24 h arimoclomol treatment groups was significantly greater than that in the corresponding control groups on and after the 14-day time point. FIG. 19b further illustrates this point.

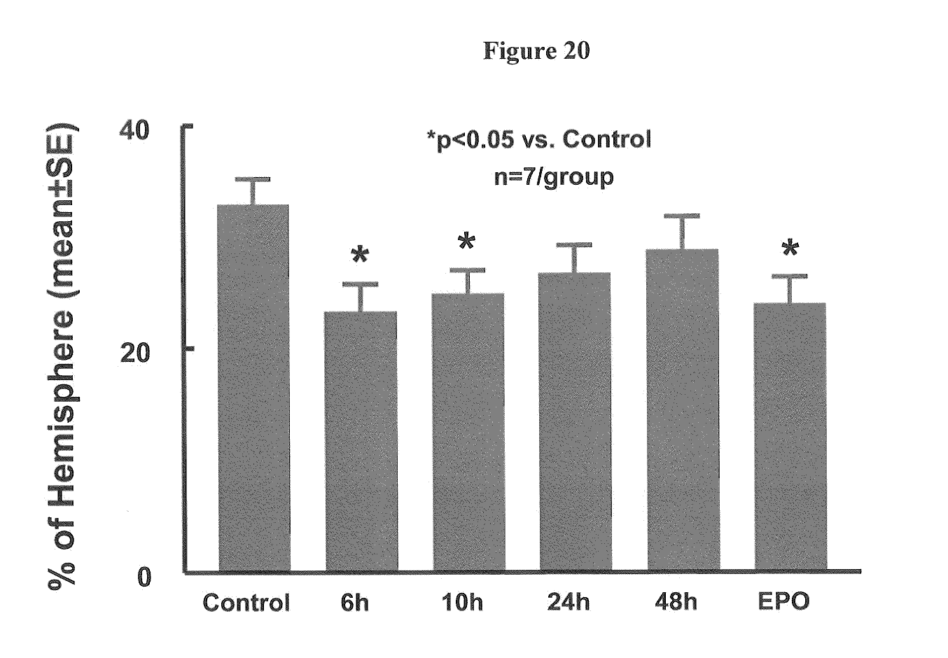

FIG. 20 shows the results of the infarct volume measured after 28 days of treatment with arimoclomol. FIG. 20 illustrates that the 6 h and 10 h arimoclomol treatment groups showed a significantly lower percentage of infarct volume compared to infarct volumes in the corresponding control groups. FIG. 20 also illustrates that the 6 h arimoclomol treatment group showed similar results as did the EPO treatment group.

In conclusion, significant differences were seen in neurological recovery following embolic stroke among the six groups. In all behavioral tests, the 6, 10 and 24 h arimoclomol treatment groups and the EPO treatment group showed significant enhancements of recovery compared to the corresponding control groups. There was also significant improvement of infarct volume seen after 28 days of treatment in the 6 h and 10 h arimoclomol treatment groups as well as with the EPO treatment group. Overall, these results indicate that arimoclomol, and compounds sharing common structural elements, will be useful as drugs that enhance functional recovery after embolic stroke.

The contents of any patents, patent applications, patent publications, or scientific articles referenced anywhere in this application are herein incorporated by reference in their entirety.

We claim:

1. A method of treating stroke comprising administering to a subject in need thereof an effective amount of a chemical compound, wherein the chemical compound is one or more of a hydroxylamine derivative represented by Formulae (I), (II) or (I′): 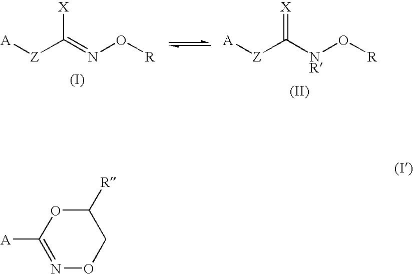
or a salt thereof or any optically active stereoisomer thereof, wherein
A is an alkyl, substituted alkyl, aralkyl, aralkyl substituted in the aryl and/or in the alkyl moiety, aryl, substituted aryl, heteroaryl or substituted heteroaryl group,
Z is a covalent bond, oxygen or ═NR³ wherein R³ is selected from the group consisting of hydro-gen, an alkyl, substituted alkyl, aryl, substituted aryl, aralkyl and aralkyl substituted in the aryl and/or in the alkyl moiety,
R is alkyl or substituted alkyl,
X in the tautomer of Formula (I) is halo or substituted hydroxy or amino, monosubstituted amino or disubstituted amino group and
X in the tautomer of Formula (II) is oxygen, imino or substituted imino group and
R′ is hydrogen, an alkyl, substituted alkyl, aryl, substituted aryl, aralkyl, aralkyl having substituted aryl or alkyl moiety, acyl or substituted acyl group,
and the compounds of Formula (I) optionally contain intramolecular ring structure of 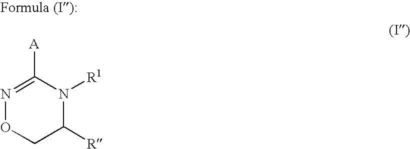
thereby reducing or preventing the symptoms associated with stroke.

2. The method according to claim 1 wherein the chemical compound is represented by Formula (I), wherein R is alkyl or substituted alkyl, and
(a) Z is covalent bond and X is halogen; or
(b) Z is covalent bond and X is a substituted hydroxy group —OQ, wherein Q is a hydrocarbon; or
(c) Z is covalent bond and X is NR¹R², wherein
R¹ and R², are independently H, linear or branched alkyl, substituted linear or branched alkyl, cycloalkyl, or
R¹ and R², together with the nitrogen atom attached thereto, form a saturated ring containing 3 to 7 members.

3. The method according to claim 2 wherein R is an ω-amino-alkyl optionally substituted on one or more of the amino or alkyl group, and the alkyl chain, which contains 3 to 8 carbon atoms, and is straight or branched, can be substituted with hydroxy or acyloxy.

4. The method according to claim 3 wherein R is an ω-amino-alkyl mono- or disubstituted on the amino, wherein the amino substituent, independently from each other are one or two straight or branched alkyl or cycloalkyl, or the two amino substituents, together with the nitrogen atom attached thereto form a 3 to 7-membered saturated hetero ring, which may contain additional hetero atom(s).

5. The method according to claim 2, wherein
Z is a chemical bond,
X is halo, and
A is aralkyl, aralkyl substituted on one or more of the aryl or alkyl moiety, aryl, substituted aryl or heteroaryl.

6. The method according to claim 5, wherein A is
(a) an unsubstituted or substituted phenylalkyl which may have one or more alkoxy substituents,
(b) phenyl,
(c) phenyl substituted with one or more of halo, alkyl, alkoxy, haloalkyl or nitro,
(d) naphthyl,
(e) N-containing heteroaryl which may be condensed with a benzene ring, or
(f) an S-containing or O-containing heteroaryl.

7. The method according to claim 2, wherein
Z is a chemical bond,
X is a substituted hydroxy of the formula —OQ, wherein Q is straight or branched and wherein Q is unsubstituted or substituted alkyl, unsubstituted aralkyl, or aralkyl substituted in one or more of the aryl or alkyl moiety, and
A is heteroaryl.

8. The method according to claim 2, wherein
Z is a chemical bond,
X is —NR¹R², wherein
R¹ and R² are independently H, a straight or branched unsubstituted alkyl, a substituted straight or branched alkyl, or cycloalkyl, or
R¹ and R², together with the N-atom adjacent thereto, form a 3 to 7-membered saturated ring, and
A is aralkyl, aralkyl substituted in one or both of the aryl or alkyl moiety, unsubstituted or substituted aryl, or heteroaryl.

9. The method according to claim 8 wherein A is
(a) phenylalkyl,
(b) phenylalkyl optionally having one or more substituents in the phenyl moiety,
(c) phenyl,
(d) phenyl substituted with one or more alkyl, halo, alkoxy, haloalkyl, nitro or acylamino,
(e) naphthyl,
(f) N-containing heteroaryl which may be condensed with a benzene ring, or
(g) an S-containing or O-containing heteroaryl.

10. The method according to claim 1 wherein the compound is represented by Formula (I), wherein
a) X is halo, Z is chemical bond and
a1) A is a group of the Formula (a): 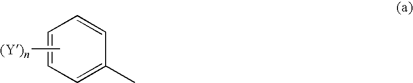
wherein Y¹ is halo, alkoxy, haloalkyl or nitro and n is 1, 2 or 3, or an O-containing heteroaryl, an S-containing heteroaryl, or an N-containing heteroaromatic group which may be condensed with a benzene ring, or the N—C₁₋₄ alkyl quaternary derivative or the N-oxide thereof,
R is a group of the Formula (b): 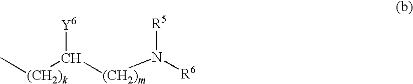
wherein R⁵ and R⁶ are independently H, straight or branched alkyl, or cycloalkyl, or R⁵ and R⁶ when taken together with the N-atom adjacent thereto form a 3 to 7-membered, saturated heterocyclic ring,
Y⁶ is —OR⁷, wherein R⁷ is H or unsubstituted or substituted alkylcarbonyl, arylcarbonyl or aminoacyl, k is 1, 2 or 3, and
m is 1, 2 or 3,
or an N—C₁₋₄ alkyl-quaternary derivative or N-oxide thereof,
with the proviso that when A is pyridyl, or a group of the Formula (a), wherein Y¹ is halo or alkoxy, R⁷ is other than H, or
a2) A is a group of the Formula (c): 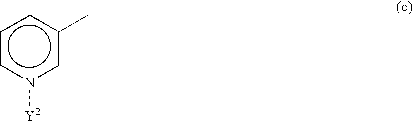
R is a group of the Formula (d): 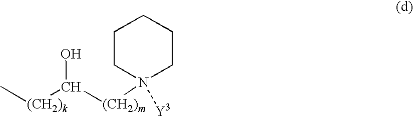
and the optional substituents Y² and Y³, one of which must be present in the molecule, are oxygen or C₁₋₄ alkyl,
k is 1, 2 or 3 and m is 1, 2 or 3 and,
when the compound is a mono- or divalent cation, the anion is one or two halide ions, or
b) A is unsubstituted or substituted aryl or an N-containing heteroaromatic group or an S-containing heteroaromatic group,
Z is chemical bond, X is OQ, wherein Q is C₁₋₄ alkyl, and
R is a group of the Formula (b): 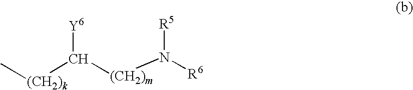
wherein R⁵ and R⁶ are independently H, straight or branched alkyl or cycloalkyl, or R⁵ and R⁶ when taken together with the N-atom adjacent thereto form a 3 to 7-membered saturated heterocyclic ring,
Y⁶ is H,
k is 1, 2 or 3, and
m is 1, 2 or 3.

11. The method according to claim 10 wherein the compound A is a group of the Formula (a) and Y¹ is C₁₋₄ haloalkyl.

12. The method according to claim 10 wherein the compound is an optically active stereoisomer of a hydroxylamine derivative wherein
X is halo,
Z is chemical bond, and
R is a group of the Formula (b), wherein R⁵ and R⁶ are independently H, straight or branched alkyl or cycloalkyl, or R⁵ and R⁶ when taken together with the N-atom adjacent thereto form a 3 to 7-membered saturated heterocyclic ring,
Y⁶ is —OR⁷ wherein R⁷ is aminoacyl,
k is 1, 2 or 3, and
m is 1, 2 or 3.

13. The method according to claim 1 wherein the compound is represented by the Formula (I), wherein
X is NR^{X}R¹, wherein R¹ and R² are independently H or unsubstituted or substituted straight or branched alkyl, or cycloalkyl, or R¹ and R², when taken together with the N-atom attached thereto, form a 3 to 7-membered hetero ring,
A is unsubstituted or substituted phenylalkyl substituted with one or more alkoxy, phenyl, phenyl substituted with one or more halo, alkyl or haloalkyl or acylamino or nitro, or an unsubstituted or substituted N-containing heteroaromatic group which may be condensed with a benzene ring, or an S-containing heteroaryl, wherein the hetero atoms may have one or more alkyl substituent(s),
Z is a chemical bond, and
R is a group of the Formula (e): 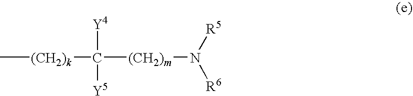
wherein R⁵ and R⁶ are independently H, straight or branched alkyl, or cycloalkyl, or
R⁵ and R⁶ when taken together with the N-atom adjacent thereto form a 3 to 7-membered saturated heterocyclic ring, which may have additional hetero atom(s) and optionally C₁₋₄ alkyl substituent(s),
Y⁴ is H or unsubstituted or substituted C₁₋₄ alkyl,
Y⁵ is H or unsubstituted or substituted C₁₋₄ alkyl or —OR⁷, wherein R⁷ is H or acyl,
k is 1, 2 or 3, and m is 1, 2 or 3,
with the proviso, that when R⁷ is H, at least one of R¹ and R² is other than H, and
when R¹ and R² are each H, R⁷ is other than H.

14. The method according to claim 10, wherein in subparagraph (a), A is furyl, thienyl, pyridyl, quinolyl or isoquinolyl.

15. The method according to claim 10, wherein in subparagraph (b), A is phenyl or pyridyl.

16. The method according to claim 13, wherein A is pyrrolyl, pyridyl, isoquinolyl, quinolyl or thienyl.

17. The method according to claim 1, wherein the compound is selected from the group consisting of:
N-[2-hydroxy-3-(1-piperidinyl) propoxy]-3 pyridine-carboximidoyl-chloride (bimoclomol),
N-[2-hydroxy-3-(1-piperidinyl)propoxy]-pyridine-1-oxide-3-carboximidoyl chloride (arimoclomol),
N-[3-(1,1-dimethylethyl)amino] 2-hydroxypropoxy]-3-trifluoromethylbenzene-carboximidoyl chloride, and
5,6-dihydro-5 (1-piperidinyl)-methyl-3-(3-pyridyl)-4H-1,2,4-oxadiazine (iroxanadine),
or a pharmaceutically acceptable salt thereof or any optically active stereoisomer thereof.

18. The method according to claim 17, wherein the compound is N-[2-hydroxy-3-(1-piperidinyl)propoxy]-pyridine-1-oxide-3-carboximidoyl chloride (arimoclomol) or a pharmaceutically acceptable salt thereof or any optically active stereoisomer thereof.

19. The method according to claim 17, wherein the compound is administered at least one hour after stroke has occurred.

20. The method according to claim 19, wherein the compound is administered at least six hours after stroke has occurred.

21. The method according to claim 20, wherein the compound is administered at least ten hours after stroke has occurred.

22. The method according to claim 21, wherein the compound is administered at least twenty four hours after stroke has occurred.

23. The method according to claim 22, wherein the compound is administered at least 72 hours after stroke has occurred.

24. The method according to claim 17, further comprising administering an additional therapeutic agent.

25. The method according to claim 17, further administering a second hydroxylamine derivative.

26. The method according to claim 25, wherein the hydroxylamine derivative is arimoclomol and the second hydroxylamine derivative is iroxanadine.

27. The method according to claim 24, wherein the additional therapeutic agent is selected from the group consisting of anti-inflammatory agents, oxygen radical scavenger, anti-pyretic agents, anti-thrombosis agents (antiplatelet agents and anticoagulants), thrombolytics, and neuroprotective agents.

28. The method according to claim 1, wherein the stroke is an embolic stroke.

29. The method according to claim 1, wherein the stroke is a thrombotic stroke.
